SEQUENCE LISTING (68 sequences)

SEQ ID NO: 1 - DNA, 6407 bp, M13 bacteriophage
  source 1..6407: mol_type = genomic DNA
aacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 2 - DNA, 6398 bp, M13 bacteriophage
  source 1..6398: mol_type = genomic DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatctgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaagcttactaaaatatatttgaaaaagttttcacgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggtaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggacgtt

SEQ ID NO: 3 - DNA, 6428 bp, synthetic construct
  source 1..6428: mol_type = other DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatctgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctcttttttagttattagaggtggtggttctggtggcggctctgagggtggtggctctgagtttttagttattagaggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaagcttactaaaatatatttgaaaaagttttcacgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggtaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggacgtt

SEQ ID NO: 4 - protein, 5 aa, synthetic construct
FLVIR

SEQ ID NO: 5 - DNA, 1218 bp, synthetic construct
  source 1..1218: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 6 - protein, 406 aa, synthetic construct
AETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 7 - DNA, 55 bp, synthetic construct
  source 1..55: mol_type = other DNA
ggtggtggctctgagggtggcggttctgagggtggcggttctgagggtggcggta

SEQ ID NO: 8 - protein, 18 aa, synthetic construct
GGGSEGGGSEGGGSEGGG

SEQ ID NO: 9 - DNA, 117 bp, synthetic construct
  source 1..117: mol_type = other DNA
ggcggcggctctggtggtggttctggtggcggctctgagggtggtggctctgagggtggcggttctgagggtggcggctctgagggaggcggttccggtggtggctctggttccggt

SEQ ID NO: 10 - protein, 39 aa, synthetic construct
GGGSGGGSGGGSEGGGSEGGGSEGGGSEGGGSGGGSGSG

SEQ ID NO: 11 - DNA, 1218 bp, synthetic construct
  source 1..1218: mol_type = other DNA
gccgagacagtggagagctgcctggccaagtcgcacaccgagaacagcttcaccaatgtttggaaggatgataagaccctggaccgctatgccaattacgaaggttgcttatggaacgcaaccggtgtggttgtgtgcacaggcgatgagacccaatgctatggcacctgggtgccgatcggtctggcaattccggagaacgaaggcggaggtagcgaaggaggtggaagtgaaggcggaggatcggaagggggtggcacaaagccaccagaatatggagacaccccgattccaggttacacctacattaatccgctggatggtacataccctccaggcaccgaacagaatccggcaaacccgaacccgagcctggaagaaagccaaccgctgaacacatttatgttccaaaacaaccgttttcgtaaccgtcaaggagccctgaccgtatacaccggtacagtgacccagggtacagatccggtgaagacctactatcaatatacaccggttagcagcaaggcaatgtacgatgcatattggaatggcaagtttcgtgattgtgcatttcatagcggtttcaacgaagacccgtttgtgtgcgaataccagggtcagagcagcgatttaccgcagccaccggttaacgcaggtggtggaagcggagggggaagtggcggtgggtcagaaggcggaggatcggaaggaggtgggagtgaaggagggggaagcgaaggagggggatcaggaggtggtagcggaagtggcgacttcgactacgagaagatggccaatgcaaacaaaggcgcaatgacagagaacgcagacgagaatgcactgcaaagtgatgcaaagggtaagctggacagcgttgcaaccgactatggagcagcaattgacggctttatcggagatgtcagcggtctggcgaacggcaacggagcaacaggcgacttcgcaggtagcaacagccagatggcacaggttggagatggcgacaacagtccgctgatgaacaactttcgccagtacctgccgagtctgccacaaagcgtcgagtgccgtccgtacgttttcggtgcaggcaagccgtacgagttcagcatcgactgcgataagattaatctttttcgcggagttttcgcattcctgctgtacgtggcaacgttcatgtacgttttcagcaccttcgccaatatcttacgcaacaaagaaagc

SEQ ID NO: 12 - protein, 9 aa, synthetic construct
MSTRGPTPA

SEQ ID NO: 13 - protein, 9 aa, synthetic construct
MTAPAPGLQ

SEQ ID NO: 14 - protein, 9 aa, synthetic construct
MTSSSDLRL

SEQ ID NO: 15 - protein, 9 aa, synthetic construct
LSSRTTYQG

SEQ ID NO: 16 - protein, 9 aa, synthetic construct
MTSKNTQIG

SEQ ID NO: 17 - protein, 9 aa, synthetic construct
MSHVGFETT

SEQ ID NO: 18 - protein, 9 aa, synthetic construct
MQPMGSTAS

SEQ ID NO: 19 - protein, 9 aa, synthetic construct
MTPSRLPPS

SEQ ID NO: 20 - protein, 9 aa, synthetic construct
MSKQNYHVV

SEQ ID NO: 21 - protein, 9 aa, synthetic construct
MAGYRSAVN

SEQ ID NO: 22 - protein, 9 aa, synthetic construct
MTTKHVATQ

SEQ ID NO: 23 - protein, 9 aa, synthetic construct
MTRTSTEPT

SEQ ID NO: 24 - protein, 9 aa, synthetic construct
MTTPNPKVR

SEQ ID NO: 25 - protein, 9 aa, synthetic construct
LTRQTNLEV

SEQ ID NO: 26 - protein, 9 aa, synthetic construct
SSRPPIVTP

SEQ ID NO: 27 - protein, 9 aa, synthetic construct
YTRPMSAPN

SEQ ID NO: 28 - protein, 9 aa, synthetic construct
FTSPPTEPR

SEQ ID NO: 29 - protein, 9 aa, synthetic construct
MGNWTPHGT

SEQ ID NO: 30 - protein, 9 aa, synthetic construct
MTSSRDAPA

SEQ ID NO: 31 - protein, 9 aa, synthetic construct
MSRQSVHTT

SEQ ID NO: 32 - protein, 9 aa, synthetic construct
FTSQTKVAM

SEQ ID NO: 33 - protein, 9 aa, synthetic construct
MSRPSSTLL

SEQ ID NO: 34 - protein, 9 aa, synthetic construct
MSTPLDRTN

SEQ ID NO: 35 - protein, 9 aa, synthetic construct
MQMATSTPA

SEQ ID NO: 36 - protein, 9 aa, synthetic construct
MSKPTRLPV

SEQ ID NO: 37 - protein, 9 aa, synthetic construct
LTTTRSLPS

SEQ ID NO: 38 - protein, 9 aa, synthetic construct
MGSPPTYRP

SEQ ID NO: 39 - protein, 9 aa, synthetic construct
MSLKSTPHP

SEQ ID NO: 40 - protein, 9 aa, synthetic construct
MSTAPPSRT

SEQ ID NO: 41 - protein, 9 aa, synthetic construct
MTSPNIAEP

SEQ ID NO: 42 - protein, 9 aa, synthetic construct
ASKVPPSGP

SEQ ID NO: 43 - protein, 9 aa, synthetic construct
AASTRPPQL

SEQ ID NO: 44 - protein, 9 aa, synthetic construct
MSQRLSHHD

SEQ ID NO: 45 - protein, 9 aa, synthetic construct
RLAKAPPVS

SEQ ID NO: 46 - protein, 9 aa, synthetic construct
MSRTNTTVN

SEQ ID NO: 47 - protein, 9 aa, synthetic construct
MSNPLSLPA

SEQ ID NO: 48 - protein, 9 aa, synthetic construct
MSNTFHRSE

SEQ ID NO: 49 - DNA, 1248 bp, synthetic construct
  source 1..1248: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctcttttttagttattagaggtggtggttctggtggcggctctgagggtggtggctctgagtttttagttattagaggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 50 - DNA, 1233 bp, synthetic construct
  source 1..1233: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagtttttagttattagaggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagtttttagttattagaggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 51 - DNA, 1248 bp, synthetic construct
  source 1..1248: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagtttttagttattagaggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagtttttagttattagaggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 52 - DNA, 1248 bp, synthetic construct
  source 1..1248: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctcttttttagttattagaggtggtggttctggtggcggctctgagggtggtggctctgagggtggcggttctgagggtggcggctctgagggaggcggttcctttttagttattagaggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 53 - DNA, 1248 bp, synthetic construct
  source 1..1248: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagtttttagttattagaggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagtttttagttattagaggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 54 - DNA, 1248 bp, synthetic construct
  source 1..1248: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagtttttagttattagaggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgcttttttagttattagaggcggcggctctggtggtggttctggtggcggctctgagggtggtggctctgagggtggcggttctgagtttttagttattagaggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 55 - DNA, 1248 bp, synthetic construct
  source 1..1248: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagtttttagttattagaggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctcttttttagttattagaggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 56 - DNA, 1233 bp, synthetic construct
  source 1..1233: mol_type = other DNA
gctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagtttttagttattagaggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttcttttttagttattagaggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 57 - DNA, 8489 bp, synthetic construct
  source 1..8489: mol_type = other DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatctgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactgttaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtccggcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgaggagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggttctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaagcttactaaaatatatttgaaaaagttttcacgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaatgattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcacacaggattttcgcctgctggggcaaaccagcgtggaccgcttgctgcaactctctcagggccaggcggtgaagggcaatcagctgttgcccgtctcgctggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcttgacactttatgcttccggctcgtataatgtgtggaattgtgagcggataacaatttcacacgccaaggagacagtcataatgaaatacctattgcctacggcagccgctggattgttattactcgctgcccaaccagccatggcctaacggggggaattcggggggccctttaaagaattcgcatacgaattctttaaagggccccccgaattccccccgttataacggcggaggatctggcgagcaaaagctcattagtgaagaggatcttgagacagtggagagctgcctggccaagccgcacaccgagaacagcttcaccaatgtttggaaggatgataagaccctggaccgctatgccaattacgaaggttgcttatggaacgcaaccggtgtggttgtgtgcacaggcgatgagacccaatgctatggcacctgggtgccgatcggtctggcaattccggagaacgaaggcggaggtagcgaaggaggtggaagtgaaggcggaggatcggaagggggtggcacaaagccaccagaatatggagacaccccgattccaggttacacctacattaatccgctggatggtacataccctccaggcaccgaacagaatccggcaaacccgaacccgagcctggaagaaagccaaccgctgaacacatttatgttccaaaacaaccgttttcgtaaccgtcaaggagccctgaccgtatacaccggtacagtgacccagggtacagatccggtgaagacctactatcaatatacaccggttagcagcaaggcaatgtacgatgcatattggaatggcaagtttcgtgattgtgcatttcatagcggtttcaacgaagacccgtttgtgtgcgaataccagggtcagagcagcgatttaccgcagccaccggttaacgcaggtggtggaagcggagggggaagtggcggtgggtcagaaggcggaggatcggaaggaggtgggagtgaaggagggggaagcgaaggagggggatcaggaggtggtagcggaagtggcgacttcgactacgagaagatggccaatgcaaacaaaggcgcaatgacagagaacgcagacgagaatgcactgcaaagtgatgcaaagggtaagctggacagcgttgcaaccgactatggagcagcaattgacggctttatcggagatgtcagcggtctggcgaacggcaacggagcaacaggcgacttcgcaggtagcaacagccagatggcacaggttggagatggcgacaacagtccgctgatgaacaactttcgccagtacctgccgagtctgccacaaagcgtcgagtgccgtccgtacgttttcggtgcaggcaagccgtacgagttcagcatcgactgcgataagattaatctttttcgcggagttttcgcattcctgctgtacgtggcaacgttcatgtacgttttcagcaccttcgccaatatcttacgcaacaaagaaagctaagcaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggacgtt

SEQ ID NO: 58 - DNA, 8549 bp, synthetic construct
  source 1..8549: mol_type = other DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatctgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactgttaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtccggcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgaggagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctcttttttagttattagaggtggtggttctggtggcggctctgagggtggtggctctgagtttttagttattagaggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaagcttactaaaatatatttgaaaaagttttcacgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaatgattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcacacaggattttcgcctgctggggcaaaccagcgtggaccgcttgctgcaactctctcagggccaggcggtgaagggcaatcagctgttgcccgtctcgctggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcttgacactttatgcttccggctcgtataatgtgtggaattgtgagcggataacaatttcacacgccaaggagacagtcataatgaaatacctattgcctacggcagccgctggattgttattactcgctgcccaaccagccatggcctaacggggggaattcggggggccctttaaagaattcgcatacgaattctttaaagggccccccgaattccccccgttataacggcggaggatctggcgagcaaaagctcattagtgaagaggatcttgagacagtggagagctgcctggccaagccgcacaccgagaacagcttcaccaatgtttggaaggatgataagaccctggaccgctatgccaattacgaaggttgcttatggaacgcaaccggtgtggttgtgtgcacaggcgatgagacccaatgctatggcacctgggtgccgatcggtctggcaattccggagaacgaaggcggaggtagcgaaggaggtggaagtgaaggcggaggatcggaagggggtggcacaaagccaccagaatatggagacaccccgattccaggttacacctacattaatccgctggatggtacataccctccaggcaccgaacagaatccggcaaacccgaacccgagcctggaagaaagccaaccgctgaacacatttatgttccaaaacaaccgttttcgtaaccgtcaaggagccctgaccgtatacaccggtacagtgacccagggtacagatccggtgaagacctactatcaatatacaccggttagcagcaaggcaatgtacgatgcatattggaatggcaagtttcgtgattgtgcatttcatagcggtttcaacgaagacccgtttgtgtgcgaataccagggtcagagcagcgatttaccgcagccaccggttaacgcaggtggtggaagctttttagttattagaggagggggaagtggcggtgggtcagaaggcggaggatcggaatttttagttattagaggaggtgggagtgaaggagggggaagcgaaggagggggatcaggaggtggtagcggaagtggcgacttcgactacgagaagatggccaatgcaaacaaaggcgcaatgacagagaacgcagacgagaatgcactgcaaagtgatgcaaagggtaagctggacagcgttgcaaccgactatggagcagcaattgacggctttatcggagatgtcagcggtctggcgaacggcaacggagcaacaggcgacttcgcaggtagcaacagccagatggcacaggttggagatggcgacaacagtccgctgatgaacaactttcgccagtacctgccgagtctgccacaaagcgtcgagtgccgtccgtacgttttcggtgcaggcaagccgtacgagttcagcatcgactgcgataagattaatctttttcgcggagttttcgcattcctgctgtacgtggcaacgttcatgtacgttttcagcaccttcgccaatatcttacgcaacaaagaaagctaagcaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggacgtt

SEQ ID NO: 59 - DNA, 1218 bp, synthetic construct
  source 1..1218: mol_type = other DNA
gctgaaactgttgaaagttgtttagcaaaatcccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 60 - protein, 406 aa, synthetic construct
AETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 61 - DNA, 1206 bp, synthetic construct
  source 1..1206: mol_type = other DNA
gctgaaactgttgaaagttgtccggcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgaggagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggttctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtct

SEQ ID NO: 62 - protein, 406 aa, synthetic construct
AETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 63 - protein, 406 aa, synthetic construct
AETVESCPAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 64 - DNA, 1218 bp, synthetic construct
  source 1..1218: mol_type = other DNA
gccgagacagtggagagctgcctggccaagtcgcacaccgagaacagcttcaccaatgtttggaaggatgataagaccctggaccgctatgccaattacgaaggttgcttatggaacgcaaccggtgtggttgtgtgcacaggcgatgagacccaatgctatggcacctgggtgccgatcggtctggcaattccggagaacgaaggcggaggtagcgaaggaggtggaagtgaaggcggaggatcggaagggggtggcacaaagccaccagaatatggagacaccccgattccaggttacacctacattaatccgctggatggtacataccctccaggcaccgaacagaatccggcaaacccgaacccgagcctggaagaaagccaaccgctgaacacatttatgttccaaaacaaccgttttcgtaaccgtcaaggagccctgaccgtatacaccggtacagtgacccagggtacagatccggtgaagacctactatcaatatacaccggttagcagcaaggcaatgtacgatgcatattggaatggcaagtttcgtgattgtgcatttcatagcggtttcaacgaagacctgtttgtgtgcgaataccagggtcagagcagcgatttaccgcagccaccggttaacgcaggtggtggaagcggagggggaagtggcggtgggtcagaaggcggaggatcggaaggaggtgggagtgaaggagggggaagcgaaggagggggatcaggaggtggtagcggaagtggcgacttcgactacgagaagatggccaatgcaaacaaaggcgcaatgacagagaacgcagacgagaatgcactgcaaagtgatgcaaagggtaagctggacagcgttgcaaccgactatggagcagcaattgacggctttatcggagatgtcagcggtctggcgaacggcaacggagcaacaggcgacttcgcaggtagcaacagccagatggcacaggttggagatggcgacaacagtccgctgatgaacaactttcgccagtacctgccgagtctgccacaaagcgtcgagtgccgtccgtttgttttcggtgcaggcaagccgtacgagttcagcatcgactgcgataagattaatctttttcgcggagttttcgcattcctgctgtacgtggcaacgttcatgtacgttttcagcaccttcgccaatatcttacgcaacaaagaaagc

SEQ ID NO: 65 - DNA, 1218 bp, synthetic construct
  source 1..1218: mol_type = other DNA
gccgaaactgttgaaagttgtccggcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 66 - protein, 402 aa, synthetic construct
AETVESCPAKPHTENSFTNVWKDDKTLDRYANYEGCLWNATGVVVCTGDETQCYGTWVPIGLAIPENEGGGSEEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 67 - DNA, 1218 bp, synthetic construct; identical to SEQ ID NO: 11
  source 1..1218: mol_type = other DNA

SEQ ID NO: 68 - protein, 406 aa, synthetic construct
AETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPYVFGAGKPYEFSIDCDKINLFRGVFAFLLYVATFMYVFSTFANILRNKES

(19) United States
(12) Patent Application Publication
(10) Pub. No.: US 2025/0163404 A1
(43) Pub. Date: May 22, 2025

(54) PHAGE DISPLAY-BASED CELL-PENETRATING PEPTIDE DISCOVERY PLATFORM AND METHODS OF MAKING AND USING THE SAME

(71) Applicant: Eli Lilly and Company, Indianapolis, IN (US)

(72) Inventors: Sepideh AFSHAR, Del Mar, CA (US); Jinsha LIU, San Diego, CA (US)

(21) Appl. No.: 18/839,228

(22) PCT Filed: Feb. 16, 2023
(86) PCT No.: PCT/US2023/062714

§ 371 Date: Aug. 16, 2024

Related U.S. Application Data
(60) Provisional application No. 63/311,270, filed on Feb. 17, 2022.

(51) Int. Cl. C12N 15/10; C40B 40/02; G01N 33/68

(52) U.S. Cl. CPC C12N 15/1037; C40B 40/02; G01N 33/6845

(57) ABSTRACT

Engineered bacteriophages are disclosed that include modifications in a pIII surface coat protein, especially in at least one of a GS1 and GS2 linker to include a peptidase recognition amino acid sequence therein. Also disclosed are methods of using such engineered bacteriophage for discovering novel cell penetrating peptides (CPPs). Novel CPPs likewise are disclosed.

The disclosure relates generally to biology and protein engineering, and more particularly it relates to phage display technologies, especially engineered M13 bacteriophage vectors that include one or more cathepsin-cleaving substrates therein, especially in a glycine/serine-rich (GS)1 linker and/or GS2 linker of protein III (pIII) for use as a novel cell-penetrating peptide (CPP) discovery platform.

RNA interference (RNAi) is a process by which double-stranded RNA (dsRNA) is used to silence gene expression. RNAi is induced by short (<30 nucleotide) double

stranded RNA (“dsRNA”) molecules which are present in the cell (Fire, et al., 1998, Nature 391:806-811). These short dsRNA molecules called “short interfering RNA” or “siRNA,” cause the destruction of messenger RNAs (“mRNAs”) which share sequence homology with the siRNA (Elbashir, et al., 2001, Genes Dev, 15:188-200). It is believed that one strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. The siRNA is apparently recycled much like a multiple-turnover enzyme, with 1 siRNA molecule capable of inducing cleavage of approximately 1000 mRNA molecules. siRNA-mediated RNAi degradation of an mRNA is therefore more effective than currently available technologies for inhibiting expression of a target gene.

Successful, active transport of therapeutic agents and/or carriers of such therapeutic agents to intracellular targets requires cell membrane translocation. Despite its selective permeability to compounds and molecules essential to cell function and survival, the cell membrane is a particularly daunting barrier. As there are three to four times more intracellular targets than cell surface targets for therapeutic agents, many delivery systems have been developed to help therapeutic agents such as peptides and siRNA cross the cell membrane and reach their intracellular target.

One such delivery system for therapeutic agents is cell-penetrating peptides (CPPs), which are versatile delivery vehicles that cross the cell membrane (see, for example, Peraro, L. and Kritzer, J. A. Emerging methods and design principles for cell-penetrant peptides. Angew. Chem. Int. Ed. Engl., 57, 11868-11881 (2018)) and are often used to carry various therapeutic cargoes such as antibodies, siRNAs and nanoparticles that are cell-impermeable into the intracellular domain which harbors about two thirds of human proteome (Overington, Al-Lazikani, & Hopkins, 2006). More specifically, CPPs are a family of short peptides, typically 5-39 amino acids in length, and often are cationic, amphipathic or hydrophobic. Unfortunately, many CPPs show poor uptake efficiency and are mainly trapped in endosomal vesicles when carrying cargos, leading to lysosome degradation. Difficulties in discriminating cytoplasmic uptake from endosomally trapped molecules have hampered the identification of true CPPs for therapeutic purposes.

Known CPP discovery and penetration measurement methods commonly require dyes and tags on CPPs, as well as include complex mammalian cell engineering for intracellular detection by microscopy or flow cytometry. Disadvantages of current cellular uptake studies include confounding effects of conjugated dyes and tags and frequent endosomal trapping with subsequent degradation.

Despite the existence of CPP discovery and penetration measurement methods, there is a need for additional CPP discovery platforms for screening and discovering CPPs with improved uptake efficiency and decreased lysosome degradation (i.e., true cytosolic internalization).

Accordingly, new CPPs are being sought that have improved cytosolic uptake efficiency and with decreased lysosome localization and which are effective for targeted delivery of therapeutic agents including peptides, polypeptides and oligonucleotides to the cytosol. To address this need, the present inventors devised an elegantly engineered phage-based CPP discovery platform that includes a library of engineered phage, as well as methods of using the phage library to efficiently identify novel and surprisingly effective CPPs. More specifically, the present disclosure is based, in part, on development of an engineered M13 bacteriophage having a modified pIII that is susceptible to lysosomal proteases and/or peptidases (including, but not limited to, one or more cathepsins). As shown herein, the modified pIII loses its ability to infect bacteria after exposure to lysosomal peptidases as the N1 and N2 domains are removed upon lysosomal peptidase digestion, which can be exploited to screen for putative CPPs that penetrate to the cytosolic domain by skipping the lysosomal localization (i.e., the CPP reaches cytosolic localization by direct-translocation or via endosomal avoidance). Notably, subsequent mechanism of action studies revealed that CPPs identified using the engineered phage-based CPP discovery platform disclosed herein enter the cell via a unique route. Thus, the CPP discovery platform disclosed herein offers a novel highly efficient approach for high-throughput discovery of cell-type-selective CPPs with sequences vastly different than traditional cell penetrating peptides.

Accordingly, the present disclosure first describes engineered M13 bacteriophages, where the engineered phages include at least a modified pIII, and where the modified pIII includes at least one exogenous peptidase recognition amino acid sequence that functions as a universal or a cell-type specific peptidase-cleaving substrate, including in some embodiments a cathepsin-cleaving substrate.

In some embodiments, the present disclosure provides modified bacteriophage pIII coat proteins of the formula (from amino-terminus (N-terminus) to carboxy-terminus (C-terminus)): displayed peptide-N1-GS1-N2-GS2-CT, wherein the C-terminus of the peptide is fused to the N-terminus of N1, and wherein there is a total of between 1 to 4 exogenous peptidase recognition amino acid sequences within GS1 and GS2 of the pIII coat protein.

In some instances, the peptidase recognition amino acid sequence is inserted into at least one of a GS1 linker and a GS2 linker of pIII. In other instances, the peptidase recognition amino acid sequence is inserted into the GS1 linker or the GS2 linker, especially the GS2 linker. In certain instances, the peptidase recognition amino acid sequence is inserted into both the GS1 linker and the GS2 linker. In some instances, the peptidase recognition amino acid sequence is inserted as a single copy. In other instances, the peptidase recognition amino acid sequence may be inserted as multiple copies such as, for example, one copy, two copies or three copies of the peptidase recognition amino acid sequence. In some instances, when multiple copies of the peptidase recognition amino acid sequence are inserted into the GS1 linker and/or the GS2 linker, the peptidase recognition amino acid sequence may be identical. In other instances, when multiple copies of a peptidase recognition amino acid sequence are inserted into the GS1 linker and/or the GS2 linker, the peptidase recognition amino acid sequences may be different. In some instances, the peptidase recognition amino acid sequence is Phe-Leu-Val-Ile-Arg (i.e., FLVIR) (SEQ ID NO: 4).

In some instances, the phage is wild-type M13 having a nucleotide sequence of SEQ ID NO: 1 modified to include a nucleotide sequence that encodes at least one exogenous peptidase recognition amino acid sequence in pIII. In other instances, the phage is M13 IX104 having a nucleotide sequence of SEQ ID NO: 2 modified to include a nucleotide sequence that encodes at least one exogenous peptidase recognition amino acid sequence in pIII. In other instances, the phage is an engineered M13 IX104 having a nucleotide sequence of SEQ ID NO: 3. In some instances, at least one exogenous peptidase recognition amino acid sequence is inserted into a GS1 linker of pIII. In other instances, at least one exogenous peptidase recognition amino acid sequence is inserted into a GS2 linker of pIII. In yet other instances, at least one exogenous peptidase recognition amino acid sequence is inserted into both the GS1 linker and the GS2 linker.

In some instances, the GS1 linker initially has a nucleotide sequence of SEQ ID NO: 7 or 8. In some instances, the GS2 linker initially has a nucleotide sequence of SEQ ID NO: 9 or 10.

In some instances, the engineered pIII further includes a CPP linked thereto. In certain instances, the CPP is a known CPP. In other instances, the CPP is a putative CPP. In some instances, the putative or known CPP is a peptide of between 4 and 39 amino acid residues. In other instances, it is a peptide of about 8 or 9 amino acids.

In certain instances, the engineered bacteriophage includes a nucleotide sequence of SEQ ID NO: 3.

Second, the disclosure describes engineered pIII that include at least one exogenous peptidase recognition amino acid sequence that functions as a universal or cell-type specific cathepsin-cleaving substrate.

In some instances, the peptidase recognition amino acid sequence is inserted into at least one of a GS1 linker and a GS2 linker of pIII. In other instances, the peptidase recognition amino acid sequence is inserted into the GS1 linker or the GS2 linker, especially the GS2 linker. In certain instances, the peptidase recognition amino acid sequence is inserted into both the GS1 linker and the GS2 linker. In some instances, the peptidase recognition amino acid sequence is inserted as a single copy. In other instances, the peptidase recognition amino acid sequence is inserted as multiple copies such as, for example, one copy, two copies or three copies. In some instances, the peptidase recognition amino acid sequence is FLVIR (SEQ ID NO: 4).

In certain instances, the engineered pIII is encoded by a nucleic acid sequence of any one of SEQ ID NOs: 49-56.

Third, the disclosure provides an engineered phage population that includes a plurality of phage clones of the engineered phage herein, where each phage clone of the plurality of phages displays the same putative CPP on pIII.

Fourth, the disclosure describes an engineered phage library that includes a plurality of phage clones of the engineered phage (i.e., phage engineered to comprise at least one exogenous peptidase recognition amino acid sequence in pIII) herein, where each phage clone of the plurality of phage also displays a putative CPP on its pIII. In some instances, an engineered phage library as described herein may have a high-complexity (e.g., >109 independent clones) or a very low complexity (e.g., between 10 to 1000 independent clones as a focused library).

Fifth, the disclosure describes methods of making an engineered bacteriophage library that include the step of modifying a pIII coat protein of a bacteriophage to comprise at least one copy of an exogenous peptidase recognition amino acid sequence comprising the amino acid sequence FLVIR as shown in SEQ ID NO: 4.

Sixth, the disclosure describes methods of screening an engineered bacteriophage library for phage clones that avoid lysosomal compartments that includes a step of exposing an engineered bacteriophage library as described herein to a target cell population for a pre-determined period of time to obtain internalized engineered bacteriophage, where the bacteriophage in the engineered bacteriophage library includes a CPP on a modified pIII as described herein. The methods also include a step of washing the target cell population to remove uninternalized engineered bacteriophage and to obtain a washed cell population. The methods also include a step of lysing the washed cell population to obtain recovered internalized engineered bacteriophage. The methods also include a step of identifying the recovered internalized engineered bacteriophage as clones that avoid lysosomal compartments in the target cell population.

In some instances, the target cell population is a eukaryotic cell population. In some instances, the eukaryotic cell population is a mammalian cell population. In certain instances, the target cell population is a population of pancreatic beta cells, adipocytes, alveolar epithelium cells, fibroblasts, skeletal muscle cells, cardiomyocytes, CHO cells, 293 cells, CaCo2 cells, or neurons, including, but not limited to, dorsal root ganglion (DRG) neurons, and hypothalamic neurons

In some instances, the CPP is a known CPP for the target cell population. In other instances, the CPP is a putative CPP for the target cell population.

In some instances, the methods optionally can include a step of amplifying the recovered internalized engineered bacteriophage prior to the identifying step.

Alternatively, the disclosure describes methods of screening an engineered bacteriophage library for phage clones that are sensitive to lysosomal enzymes that includes a step of exposing an engineered bacteriophage library as described herein to a cathepsin. The methods also include a step of identifying phage clones in the library that are cleaved or degraded as lysosomal enzyme sensitive.

In some instances, the lysosomal enzyme is a cathepsin. In some instances, the cathepsin can be cathepsin A, B, C, D, H, L and/or S.

Seventh, the disclosure describes methods of screening putative CPPs that include a step of exposing an engineered bacteriophage library as described to first target cell population for a predetermined period of time that is sufficient to allow for CPP binding and for bacteriophage internalizing, where phage clones in the engineered bacteriophage library display a distinct, putative CPP on a modified pIII as described herein. The methods also include a step of washing the first target cell population to remove uninternalized engineered bacteriophage and to obtain a washed cell population. The methods also include a step of lysing the washed cell population to obtain recovered internalized engineered bacteriophage. The methods also include a step of exposing the recovered engineered bacteriophage to a second target cell population for a predetermined period of time to penetrate the second target cell population and to amplify any recovered engineered bacteriophage that penetrated the second target cell population. The methods also include a step of identifying the CPP attached to any amplified, recovered engineered bacteriophage.

The target cell population of the CPPs disclosed herein is a eukaryotic cell population. In some instances, the eukaryotic cell population is a mammalian cell population. In certain instances, the mammalian cell population is a population of pancreatic beta cells, adipocytes, alveolar epithelium cells, fibroblasts, skeletal muscle cells, cardiomyocytes, CHO cells, 293 cells, CaCo2 cells, or neurons, including, but not limited to, dorsal root ganglion (DRG) neurons, and hypothalamic neurons.

Eighth, the disclosure describes CPPs having cytosolic localization but not lysosomal localization that include an amino acid sequence selected from any one of SEQ ID NOs: 12 to 48. Such CPPs that may be useful to facilitate active transport of therapeutic agents (and/or carriers of such therapeutic agents) including, but not limited to, peptides, proteins, lipid nanoparticles (LNPs), polymeric lipid vehicles (PLVs), oligonucleotides (e.g., mRNA, iRNA, siRNA, anti-sense oligonucleotides (ASOs), etc.), mAbs or fragments thereof, and small molecules by covalent or non-covalent bonds to intracellular targets for therapeutic and/or diagnostic purposes.

Therefore, in certain preferred embodiments, the invention provides methods of delivering therapeutic agents, including, but not limited to, interfering RNA to inhibit the expression of a target mRNA thus decreasing target mRNA levels in patients with target mRNA-related disorders.

One advantage of the platform herein is that it allows one to enrich for CPP phage clones that avoid lysosomal localization and instead have cytosolic localization.

One advantage of the platform herein is that it is free of chemical dyes and/or tags.

One advantage of the platform herein is that it can be screened in different cell types for delivering a cargo of interest.

One advantage of the platform herein is that no engineering is needed for mammalian cells.

The advantages, effects, features, and objects other than those set forth above will become more readily apparent when consideration is given to the detailed description below. Such detailed description refers to the following drawing(s), where:

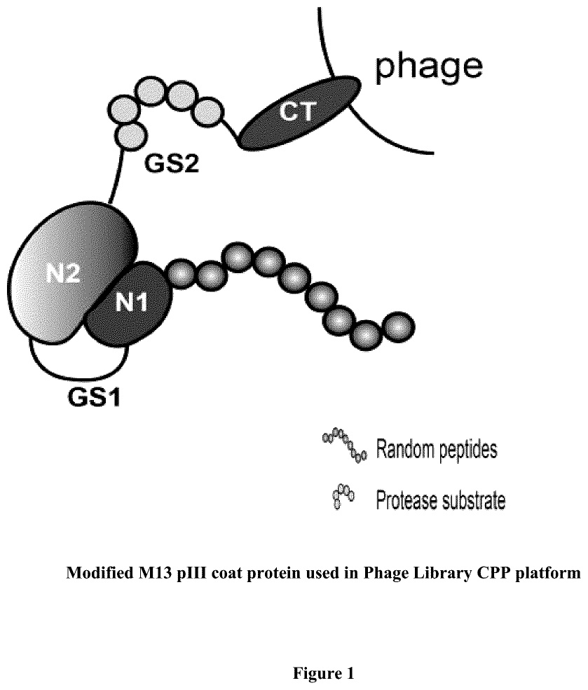

FIG. 1 illustrates a modified bacteriophage pIII coat protein as described herein. A lysosomal peptidase recognition amino acid sequence (denoted in FIG. 1 as a “protease substrate”) is engineered into the GS2 linker of M13 phage pIII coat protein. Upon entering lysosome compartments led by the random CPP peptide displayed on a particular phage in the library, the N1 and N2 domains will generally be removed by lysosomal cathepsin digestion, resulting in the loss of infectivity in a bacterial amplification step. Multiple rounds of selection may be conducted to remove lysosomal localized phage clones, and enrich for cytoplasmic up-taken phage clones. The identity of the random peptide sequence (i.e., cell penetrating peptide sequence) resulting in cytoplasmic localization is identified by sequencing analysis.

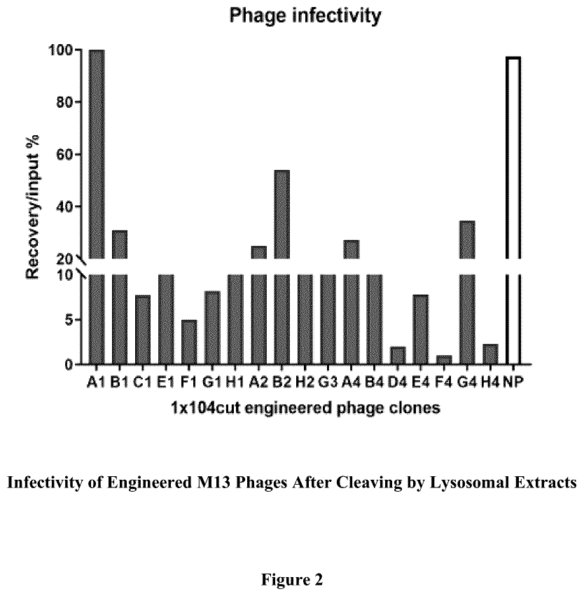

FIG. 2 shows the representative results of the infectivity of engineered M13 phage with treatment of individually isolated CHO cell lysosomal extract at pH 5.

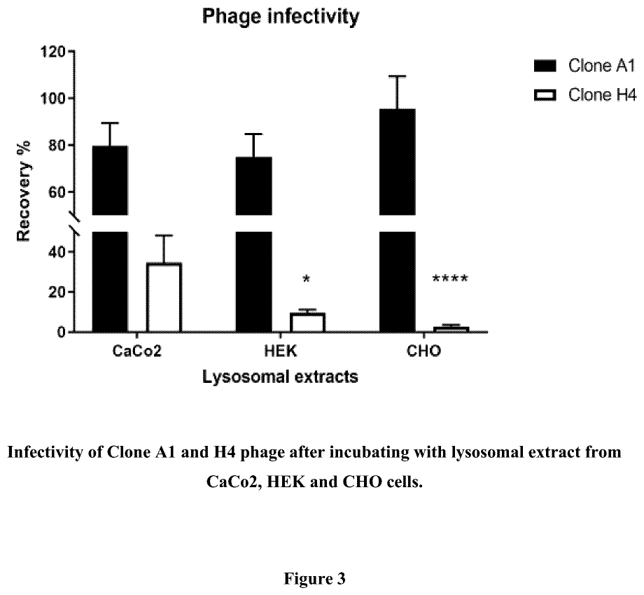

FIG. 3 shows the infectivity of Clone A1 and H4 with incubation of lysosomal extracts from CaCo2, HEK and CHO cells.

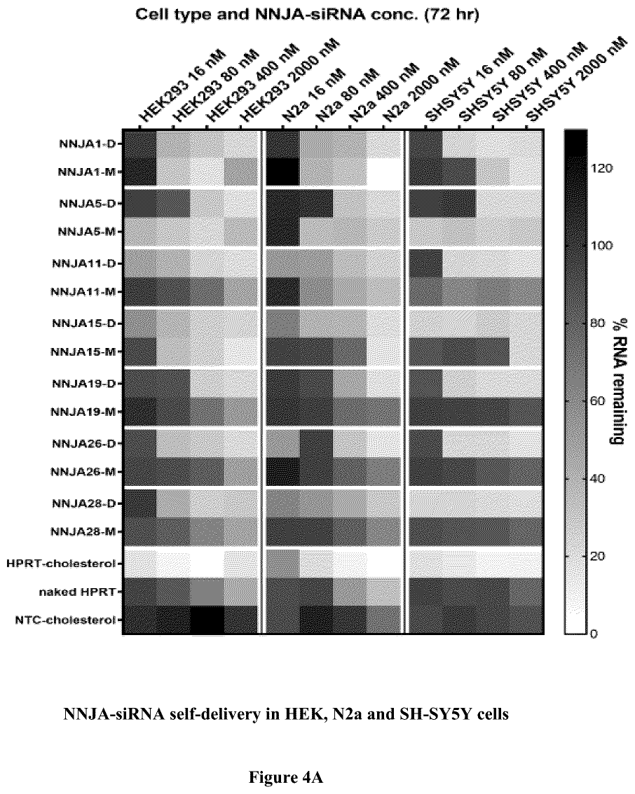

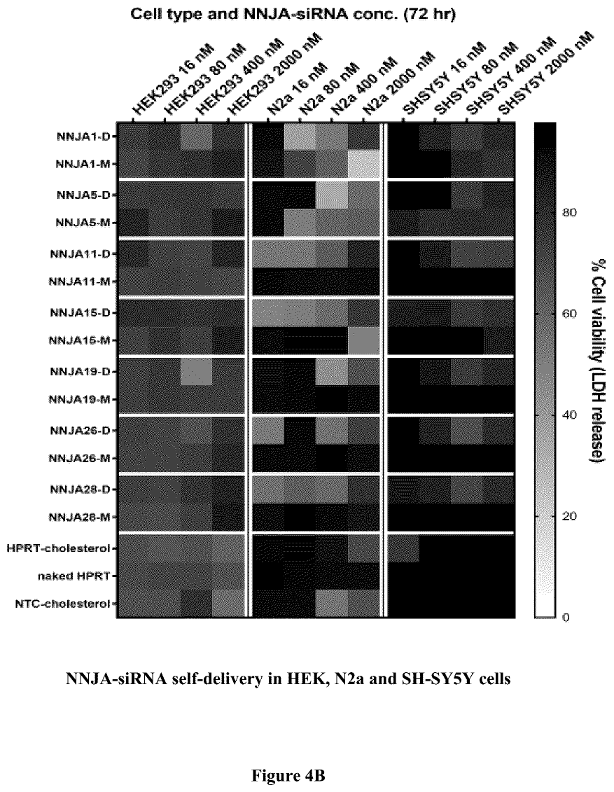

FIG. 4 shows NNJA CPP-siRNA self-delivery in HEK, N2a and SH-SY5Y cells. The percentage of RNA remaining and cell viability are evaluated. The percentage of RNA remaining inside cells is assessed by qRT-PCR at 72 hr. post treatment (FIG. 4A) and the cell viability indicated by LDH release is evaluated after compound treatment in three cell types (FIG. 4B).

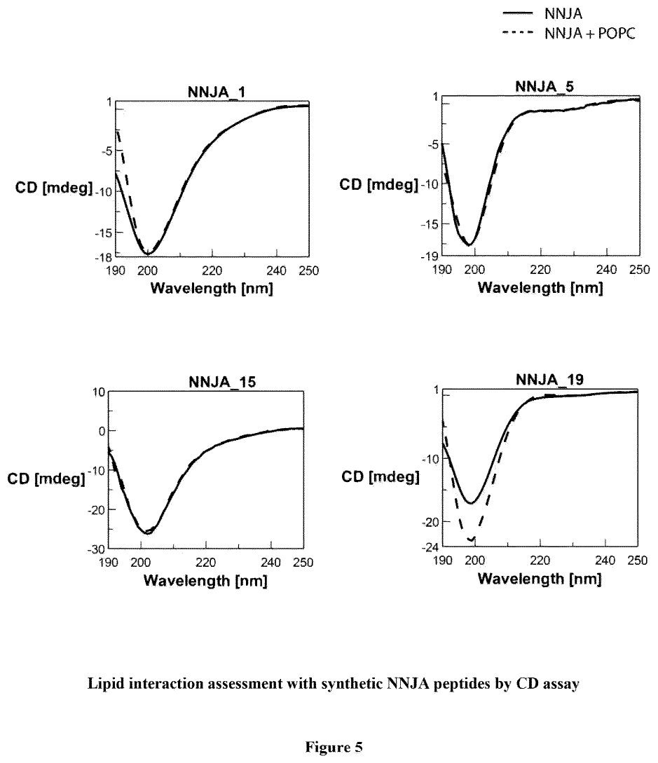

FIG. 5 shows the lipid interaction assessment with synthetic NNJA peptides by Circular Dichroism (CD) assay.

### OVERVIEW

Described herein is an engineered phage library based upon bacteriophage M13. M13 is an example of a commonly used phage for expressing heterogenous peptides and antibody fragments via phage display. Filamentous M13 assembly occurs in the bacterial inner membrane. Phage coat proteins are synthesized in the cytoplasm using bacterial protein synthetic machinery and are then directed to the periplasm by different signal peptides. Functional M13 phage particles include five types of surface coat proteins termed pIII (minor coat protein), pVI (minor coat protein), pVII (minor coat protein), pVIII (major coat protein) and pIX (minor coat protein). While all five of these surface coat proteins have been used to display exogenous peptides on the surface of M13 particles, the minor coat protein pIII is the most commonly used for anchoring peptides of interest to the phage coat surface. See, “Methods in Molecular Biology,” Vol 178, Antibody Phage Display: Methods and Protocols (O'Brien & Aitken eds.). pIII exists in 5 copies at the proximal end of the M13 phage and plays important roles in phage infectivity, assembly and stability. pIII is expressed as a 406 amino acid polypeptide and has 3 distinct regions: N1, N2 and C-terminal (CT) domains. See, Russel et al. (2002) Introduction to Phage Biology and Display, Phage Display: A Laboratory Manual; Cold Spring Harbor Lab. Press. The N1 domain participates in translocating viral DNA into a bacterial (e.g., E. coli) host during infection, while the N2 domain imparts host cell recognition by attaching to bacterial F pilus. The CT domain participates in anchoring pIII protein to the phage coat during assembly. See, Omidfar & Daneshpour (2015) Expert Opin. Drug Discov. 10:651-669. In some instances contemplated herein, pIII lacking an exogenous peptidase recognition amino acid sequence is encoded by a nucleotide sequence as shown in SEQ ID NO: 5, SEQ ID NO: 11, SEQ ID NO: 59, SEQ ID NO: 61, SEQ ID NO: 64, SEQ ID NO: 65, or SEQ ID NO: 67. Likewise, in some instances contemplated herein, pIII lacking an exogenous peptidase recognition amino acid sequence has an amino acid sequence as shown in SEQ ID NO: 6, SEQ ID NO: 60, SEQ ID NO: 62, SEQ ID NO: 63, SEQ ID NO: 66, or SEQ ID NO: 68.

The engineered phage library herein can be used to eliminate phage clones located in lysosome compartments via cellular trafficking such as endocytosis by blocking phage amplification in bacterial cells. CPP selection is enabled with this phage library by engineering an effective peptidase recognition amino acid sequence (e.g., a cathepsin recognition sequence) into at least one of a GS1 linker and/or a GS2 linker of pIII such that lysosomal proteases (e.g., cathepsins) can cleave the substrate and release N1 and N2 domains when phage clones localize in lysosome compartments. Without N1 and N2 domains, phage lose their infectivity when exposed to bacterial cells. Specifically, by depleting the lysosomal-located phage clones through multiple rounds of selection, one can enrich phage clones that can skip endocytosis and/or avoid endosome-lysosome route efficiently and localize in the cytosolic domain (see, FIG. 1).

Unless defined otherwise, all technical and scientific terms used herein have the same meaning as commonly understood by one of skill in the art to which the disclosure pertains. Although any methods and materials similar to or equivalent to those described herein can be used in the practice or testing of the phage libraries and CPPs herein, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one”.

### Definitions

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the system under study, and can be readily appreciated by one of skill in the art.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

As used herein, “cathepsin” means an aspartyl, cysteine or serine protease that typically are activated at the low pH present in lysosomes. Examples of cathepsin for use herein include, but are not limited to, cathepsin A, B, C, D, H, L and/or S. One of skill in the art understands that nucleotide and amino acid sequences for such cathepsins are readily available using publicly available databases such as, for example, GenBank and UniProt.

As used herein, “cell penetrating peptide” or “CPP” means a peptide of <40 amino acid residues that can translocate into a cell or cells without causing membrane damage and that can be use as vectors for delivering therapeutic agents and/or as carriers of such therapeutic agents to intracellular targets requires cell membrane translocation. In some embodiments, a CPP is a peptide of between 4 and 39 amino acid residues. In some embodiments, a CPP is a peptide of between 4 and 30 amino acid residues. In some embodiments, a CPP is a peptide of between 5 and 25 amino acid residues. In other embodiments, a CPP is a peptide of between 7 and 20 amino acid residues. In other embodiments, a CPP is a peptide of between 8 and 15 amino acid residues. In yet other embodiments, a CPP is a peptide of between 8 and 10 amino acid residues.

As used herein, “complementary” or “complementarity” means a structural relationship between two nucleotides, nucleosides, or nucleobases (e.g., on two opposing nucleic acids or on opposing regions of a single nucleic acid strand e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the canonical Watson-Crick manner, which means adenine pairing with thymine or uracil, and guanine pairing with cytosine, or in any other manner that allows for the formation of stable duplexes. Likewise, two nucleic acids may have regions of multiple nucleotides that are complementary with each other to form regions of complementarity.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications or substitutions of atoms other than hydroxyl at the 2′ position, including modifications or substitutions in or of the nucleobase, sugar, or phosphate group.

As used herein, “double-stranded oligonucleotide” or “ds oligonucleotide” means an oligonucleotide that is in a duplex form. The complementary base-pairing of duplex region(s) of a ds oligonucleotide can be formed between antiparallel sequences of nucleotides of covalently separate nucleic acid strands. Likewise, complementary base-pairing of duplex region(s) of a ds oligonucleotide can be formed between antiparallel sequences of nucleotides of nucleic acid strands that are covalently linked. Moreover, complementary base-pairing of duplex region(s) of a ds oligonucleotide can be formed from single nucleic acid strand that is folded (e.g., via a hairpin) to provide complementary antiparallel sequences of nucleotides that base pair together. A ds oligonucleotide can include two covalently separate nucleic acid strands that are fully duplexed with one another. However, a ds oligonucleotide can include two covalently separate nucleic acid strands that are partially duplexed (e.g., having overhangs at one or both ends). A ds oligonucleotide can include an antiparallel sequence of nucleotides that are partially complementary, and thus, may have one or more mismatches, which may include internal mismatches or end mismatches.

As used herein, “duplex” and “duplex region” in reference to nucleic acids (e.g, oligonucleotides), means a structure formed through complementary base pairing of two antiparallel sequences of nucleotides, whether formed by two covalently separate nucleic acid strands or by a single, folded strand (e.g., via a hairpin). A duplex may form despite not having full complementarity between the two strands, or when an abasic moiety is present.

As used herein, “engineered” means artificial or synthetic or modified, especially with respect to a nucleic acid sequence, amino acid sequence or organism herein. For example, “engineered” may refer to a change, such as an addition, deletion and/or substitution of a nucleic acid residue or amino acid residue with respect to a given wild-type nucleotide or amino acid sequence.

As used herein, “exogenous,” with regard to a nucleotide, oligonucleotide, polynucleotide, peptide, polypeptide or protein means a nucleic acid sequence or amino acid sequence not normally present (i.e., non-native) in the host cell or genome.

As used herein, “linker” more generally means a structure used to conjugate a molecule such as a nucleotide (e.g., oligonucleotide), peptide, or polypeptide to another molecule of the same or different kind. As noted above, certain conjugates may employ one or more linker groups. The term “linkage”, “linker”, “linker moiety, or simply “L” is used herein to refer to a linker that can be used to separate a cell penetrating peptide from an agent (e.g., a strand of an siRNA molecule, for example), or to separate a first agent from another agent or label (fluorescence label), for instance, where two or more agents are linked to form a cell penetrating peptide con. The linker may be physiologically stable or may include a releasable linker such as a labile linker or an enzymatically degradable linker (e.g., proteolytically cleavable linkers). In certain aspects, the linker may be a peptide linker. In some aspects, the linker may be a non-peptide linker or non-proteinaceous linker. In some aspects, the linker may be particle, such as a nanoparticle. The linker may be charge neutral or may bear a positive or negative charge. A reversible or labile linker contains a reversible or labile bond. In some embodiments, a linker can be “labile” or “cleavable” meaning a linker that can be cleaved (e.g., by acidic pH or enzyme). More specifically, a labile bond is a covalent bond that is less stable (thermodynamically) or more rapidly broken (kinetically) under appropriate conditions than other non-labile covalent bonds in the same molecule. Cleavage of a labile bond within a molecule may result in the formation of two molecules. For those skilled in the art, cleavage or lability of a bond is generally discussed in terms of half-life (ti/2 of bond cleavage (the time required for half of the bonds to cleave). Thus, labile bonds encompass bonds that can be selectively cleaved more rapidly than other bonds in a molecule. Appropriate conditions are determined by the type of labile bond and are well known in organic chemistry. A labile bond can be sensitive to pH, oxidative or reductive conditions or agents, temperature, salt concentration, the presence of an enzyme (such as esterases, including nucleases, and proteases), or the presence of an added agent. For example, increased or decreased pH is the appropriate conditions for a pH-labile bond. In other embodiments, a linker can be “stable” or “non-cleavable”meaning a linker that is not cleaved in physiological conditions. In some embodiments, a linker is used to conjugate a therapeutic agent to a targeting ligand or a delivery moiety.

As used herein, “glycine/serine-rich 1 linker” or “GS1 linker” means a first of two GS linkers in pIII, which is located between the N-terminal 1 (N1) domain and N-terminal 1 (N2) domain.

As used herein, “glycine/serine-rich 2 linker” or “GS2 linker” means a second of two GS linkers in pIII, which is located between the N2 domain and C-terminal (CT) domain.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

As used herein, “modulate,” “modulating,” and the like means that expression of a target gene, or level of a RNA molecule encoding a target protein or a protein subunit, or activity of a protein or protein subunit is upregulated or downregulated, such that expression, level or activity is greater than or less than that observed in the absence of the oligonucleotide. For example, “modulate” with regard to siRNA can mean to inhibit or downregulate expression of a target gene or its protein product. Likewise, “modulate” with regard to saRNA can mean to stimulate or upregulate expression of a target gene or its protein product.

As used herein, the term “NNJA” or “Ninja” in reference to CPPs, the amino acid sequences encoding the CPPs, or the nucleic acids sequences encoding the CPP amino acid sequences means that the CPPs and/or the amino acid or nucleic acid sequences encoding the CPPs were identified from use of the engineered phage-based CPP discovery platform disclosed herein. Likewise, the term “NNJA” or “Ninja” may be used to refer to the engineered phage-based CPP discovery platform disclosed herein in addition to the CPPs identified and/or characterized with such platform.

As used herein, “nucleotide” means an organic compound having a nucleoside (a nucleobase, for example, adenine, cytosine, guanine, thymine, or uracil; and a pentose sugar, for example, ribose or 2′-deoxyribose) and a phosphate group. A “nucleotide” can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length. An oligonucleotide may be single-stranded (ss) or double stranded (ds). An oligonucleotide may or may not have duplex regions.

As used herein, “overhang” means a terminal nucleotide(s) resulting from one strand or region extending beyond the terminus of a complementary strand with which the one strand or region forms a duplex. An overhang may include one or more unpaired nucleotides extending from a duplex region at the 5′ terminus or 3′ terminus of a ds oligonucleotide. The overhang can be a 3′ or 5′ overhang on the antisense strand or sense strand of a ds oligonucleotides.

As used herein, “reduced expression,” and with respect to a gene means a decrease in the amount or level of RNA transcript or protein encoded by the gene and/or a decrease in the amount or level of activity of the gene in a cell, a population of cells, a sample, or a subject, when compared to an appropriate reference (e.g., a reference cell, population of cells, sample, or subject). For example, introducing an oligonucleotide herein (e.g., an oligonucleotide having an antisense strand having a nucleotide sequence that is complementary to a nucleotide sequence) into a cell may result in a decrease in the amount or level of mRNA, protein, and/or activity (e.g., via degradation of mRNA by the RNAi pathway) when compared to a cell that is not treated with the ds oligonucleotide. Similarly, and as used herein, “reducing expression” means an act that results in reduced expression of a gene. Specifically, and as used herein, “reduction of expression” means a decrease in the amount or level of mRNA, protein, and/or activity in a cell, a population of cells, a sample, or a subject when compared to an appropriate reference (e.g., a reference cell, population of cells, tissue, or subject).

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “synthetic” refers to a nucleic acid or other compound that is artificially synthesized (e.g., using a machine such as, for example, a solid phase nucleic acid synthesizer) or that is otherwise not derived from a natural source (e.g., a cell or organism) that normally produces the nucleic acid or other compound.

As used herein, “M13” means an F-specific filamentous (Ff) phage that is a member of the family of filamentous bacteriophage. M13 is a circular, single-stranded (ss) DNA of 6407 nucleotides. One nucleotide sequence for M13 can be as provided in NCBI Ref. Seq. No. V00604.2 (SEQ ID NO: 1). Another nucleotide sequence for M13 is M13 IX104 (SEQ ID NO: 2). One of skill in the art, however, understands that additional examples of M13 nucleotide and amino acid sequences are readily available using publicly available databases such as, for example, GenBank and UniProt.

As used herein, “pIII” or “pIII coat protein” means a M13 bacteriophage surface coat protein of about 406 amino acid residues (see, e.g., SEQ ID NOs: 6, 60, or 62) that includes three major domains linked by two GS linkers: N1, N2 and CT domains.

As used herein, a “peptidase recognition amino acid sequence” is a sequence of about 5-9 amino acids long, more typically, about 4-7 amino acids long, that is involved in peptidase recognition and cleavage of a peptide having said sequence. Numerous examples of peptidase recognition amino acid sequences including those known to be recognized and cleaved by cathepsins are well known in the prior art and thus, do not need detailed description herein.

As used herein, the terms “protease” and “peptidase” are used interchangeably.

As used herein, “aRNA,” “aRNA agent,” “RNAa,” “RNAa agent” and “RNA activating agent” means an agent that contains RNA and that mediates the targeted activation of a promoter or other non-coding transcript of an RNA transcript via an RNA-induced transcriptional activation (RITA) complex pathway. The aRNA activates, increases, modulates, or upregulates expression in a cell.

As used herein, “iRNA,” “iRNA agent,” “RNAi,” “RNAi agent” and “RNA interference agent” means an agent that contains RNA and mediates the targeted cleavage of a RNA transcript via RNA interference, e.g., through an RNA-induced silencing complex (RISC) pathway. In some embodiments, the RNAi agent has a sense strand and an antisense strand, and the sense strand and the antisense strand form a duplex. In some embodiments, the sense and antisense strands of RNAi agent are 21-23 nucleotides in length. In other embodiments, the sense and antisense strands can be longer, for example 25-30 nucleotides in length, in which case the longer RNAi sequences are first processed by the Dicer enzyme. The iRNA attenuates, inhibits, modulates, or reduces expression in a cell.

As used herein, the terms “small interfering RNA (siRNA)”, “siRNA molecule” or “siRNA” are used interchangeably and refer to small inhibitory RNA duplexes that induce the RNA interference (RNAi) pathway. As used herein, these molecules can vary in length (generally 15-30 base pairs plus optionally overhangs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, and unless otherwise specified also includes single strands that can form hairpin structures comprising a duplex region, such as short-hairpin RNAs (“shRNA”). Thus, in some embodiments, the polynucleotide is a shRNA molecule, which means a molecule of double-stranded RNA, typically 20-24 base pairs in length, similar to miRNA, and operating within the RNA interference (RNAi) pathway. It is intended to interfere with the expression of specific genes with complementary nucleotide sequences by degrading mRNA after transcription, preventing translation. Small interfering RNA may also be referred to in the art as short interfering RNA or silencing RNA, for example.

As used herein, “subject” means any mammal, including cats, dogs, mice, rats, and primates, and humans. Preferably subject means humans. Moreover, “individual” or “patient” may be used interchangeably with “subject.”

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a nucleic acid or vector or composition for treatment of a disease or condition in a patient, particularly in a human. Also, consider additional disclosure to achieve a desired efficacy or outcome depending on what data we have and our draft label language.

As used herein, “vector” means a nucleic acid molecule capable of transporting another nucleic acid sequence (or multiple nucleic acid sequences) to which it has been ligated into a host cell or genome. One type of vector is a “plasmid,” which refers to a circular DNA loop, typically double-stranded (ds), into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication). Moreover, certain vectors are capable of directing the expression of genes (e.g., genes encoding an exogenous peptide or protein of interest) to which they are operatively linked when combined with appropriate control sequences such as promoter and operator sequences and replication initiation sites. Such vectors are commonly referred to as “expression vectors” and may also include a multiple cloning site for insertion of the gene encoding the protein of interest. Alternatively, the gene encoding the peptide or protein of interest may be introduced by site-directed mutagenesis techniques such as Kunkel mutagenesis. See, e.g., Handa et al., Rapid and Reliable Site-Directed Mutagenesis Using Kunkel's Approach, Methods in Molecular Biology, vol 182: In Vitro: Mutagenesis Protocols, 2nd Ed.).

### Compositions

The compositions herein include an engineered bacteriophage, especially a M13-based engineered bacteriophage. General details on M13 and phage display can be found in Intl. Patent Application Publication No. WO 2017/091467, for example.

The compositions herein also include engineered pIII coat proteins.

The compositions herein also include an engineered phage library, especially an M13-based engineered bacteriophage library. One skilled in the art would recognize that the engineered phage libraries of the type disclosed herein can be created having high diversity with respect to the putative CPPs being screened (e.g., primary library) or lower diversity with respect to the putative CPPs being screened or novel CPPs being optimized (e.g., secondary or enriched libraries) for a particular target cell population. In some instances, the diversity of an engineered phage library as disclosed herein

The compositions disclosed herein include novel CPPs. In some embodiments, the novel CPP is a peptide of between 2 and 10 amino acid residues. In other embodiments, the CPP is a peptide of between 5 and 10 amino acid residues. In yet other embodiments, a CPP is a peptide of between 8 and 10 amino acid residues. The compositions here also include CPPs, especially CPPs having 9 amino acid residues.

### Methods

The methods herein include methods of making engineered bacteriophage, especially M13-based engineered bacteriophages and libraries including the same.

Kunkel mutagenesis is well known in the art and need not be exhaustively described herein. See, e.g., Handa et al. (2002), “Rapid and Reliable Site-Directed Mutagenesis Using Kunkel's Approach” In: In Vitro Mutagenesis Protocols. Methods in Molecular Biology, vol 182. (Braman ed., Humana Press, Totowa, NJ).

The methods herein also include methods of screening for engineered bacteriophages that can avoid lysosomal localization.

In some instances, the method of screening an engineered bacteriophage or an engineered bacteriophage library for bacteriophages that are sensitive to lysosomal enzymes or that can avoid lysosomal localization, comprises the steps of:
(a) providing a library of engineered bacteriophage as disclosed herein;
(b) exposing the engineered bacteriophage library to a lysosomal enzyme for a predetermined period of time to obtain cleaved engineered bacteriophages and uncleaved engineered bacteriophages; and
(c) identifying bacteriophages that are cleaved by the lysosomal enzyme as sensitive or those that avoid lysosomal localization base on not being cleaved.

In some instances, the lysosomal enzyme is a cathepsin such as, for example, cathepsin A, B, C, D, H, L and S.

In some instances, the methods provided herein includes methods of screening putative cell-penetrating peptides (CPPs) for a specific type of cell, the method comprising the steps of:
(a) providing an engineered bacteriophage library of any one of Claims 14-15;
(b) exposing the engineered bacteriophage library to a first target cell population for a predetermined period of time to obtain internalized engineered bacteriophage;
(c) washing the first target cell population to remove uninternalized engineered bacteriophage and to obtain a washed target cell population;
(d) lysing the washed first target cell population and obtaining recovered internalized engineered bacteriophage;
(e) exposing the recovered internalized engineered bacteriophage to a second target cell population for a predetermined period of time to infect the second target cell population and to obtain amplified, recovered internalized engineered bacteriophage; and
(f) identifying the amplified, recovered engineered bacteriophage for clones that avoided lysosomal compartments in the first target cell population.

In some instances, the first target cell population is a eukaryotic cell population.

In some instances, the first target cell population is a mammalian cell population.

In some instances, the first target cell population is selected from the group consisting of pancreatic beta cells, adipocytes, alveolar epithelium cells, fibroblasts, skeletal muscle cells, cardiomyocytes, CHO cells, 293 cells, CaCo2 cells, or neurons, including, but not limited to, dorsal root ganglion (DRG) neurons, and hypothalamic neurons.

The methods herein also include methods of using engineered bacteriophages or libraries herein to screen for putative CPPs.

The methods may also include a step of exposing the recovered engineered bacteriophage to a second target cell population for a predetermined period of time to select against the second target cell population for internalization and to amplify any recovered engineered bacteriophage that penetrate the second target cell population. When a second target cell type is involved, one skilled in the art would recognize that there are many useful selection strategies possible depending on the properties desired in any novel CPP. In some instances, for example, a first and second target cell population may be co-targeted for internalization by a positive selection against the first target cell population and then taking the recovered internalized peptide-phage to further select against the second target cell population for internalization. In other instances, one skilled in the art may counter-select against a first target cell population (negative selection), and take the peptide-phage that remain outside the cells, and select against a second target cell population for internalization (positive selection). In other instances, screening methods may include positive selection against a first and second target cell population in parallel arms for internalization, then compare the peptide hits for either subtraction or consensus.

In some instances, the first and second target cell populations are eukaryotic cell populations.

In some instances, the first and second target cell populations are mammalian cell populations.

In some instances, the first and second target cell populations are each selected from the group consisting of pancreatic beta cells, adipocytes, alveolar epithelium cells, fibroblasts, skeletal muscle cells, cardiomyocytes, CHO cells, 293 cells, CaCo2 cells, or neurons, including, but not limited to, dorsal root ganglion (DRG) neurons, and hypothalamic neurons.

The methods also include a step of exposing the recovered engineered bacteriophage to a bacterial cell population for a predetermined period of time to infect the bacterial cell population and to amplify any recovered engineered bacteriophage that infected a target cell population.

The following non-limiting examples are offered for purposes of illustration, not limitation.

### Generating the Engineered M13 Phage Library

### Example 1: Engineering Cathepsin-Cleavable Substrates into GS1 and/or GS2 Linker of Bacteriophage pIII Coat Protein

### Methods:

Cells and reagents: Chinese hamster ovary (CHO-2F9 in-house; CHO) cells are grown in suspension with medium prepared in-house (M9195+12 mM L-glutamine) in 5% CO₂ at 37° C. Expi293 (293; Life Technologies) cells also are maintained as a suspension in culture medium (Cat. No. A14351-01; Gibco) in 8% CO₂ at 37° C. Adherent Colon carcinoma (CaCo2; in-house) cells are cultured in Dulbecco's Modified Eagle's Medium (DMEM) supplemented with L-glutamine, 10% heat-inactivated (HI) FBS, 1 mM sodium pyruvate and 25 mM HEPES at 5% CO₂ at 37° C. Adherent HEK293 (HEK) cells are grown in Minimum Essential Medium (MEM) supplemented with 10% HI FBS, 1× non-essential amino acids, 1 mM sodium pyruvate, and 0.075% sodium bicarbonate and used for microscopy imaging and cytotoxicity purpose. If not specified, cell culture reagents are purchased from Gibco.

Antibodies: anti-EEA1 (Cat. No. ab2900; Abcam); anti-LAMP1 (Cat. No. ab24170; Abcam).

For confocal imaging, anti-M13-Alexa 647 (in-house), anti-LAMP1 (Cat. No. 9091; Cell Signaling); anti-F-actin-DyLight488 (Cat. No. PI21833; ThermoFisher); DAPI (Cat. No. D1306; Invitrogen); Alexa Fluor 488, Alexa Fluor 568 and Alexa Fluor 647-coupled fluorescent secondary antibodies (Life Technologies).

Subcellular fractionation: cytosolic and endosomal extraction are prepared according to manufactures' protocols from ThermoFisher Scientific (Cat. No. 89842) and Invent Biotechnologies (Cat. No. #ED-028), respectively. The starting cell number is 5×10⁶ cells for one cytosolic extraction, and 3×10⁷ cells for one endosome extraction. Lysosomal isolation from different cell types are optimized based on an Abcam kit (Cat. No. ab234047) for homogenization step and increased isolation scale. The starting cell number for one lysosomal isolation is 2×10⁸ cells.

Cathepsin enzymatic cleavage assay: 6 fluorogenic peptide substrates are purchased from R&D Systems, Bachem, or Chemimpex. Cathepsin B and L share the same fluorogenic peptide-substrate, and the other 5 cathepsins recognize and cleave a specific fluorogenic peptide-substrate. The corresponding peptide substrate for each cathepsin is as follows: cathepsin A (Cat. No. ES005; R&D Systems), cathepsin B/L (Cat. No. ES008; R&D Systems), cathepsin C (Cat. No. I-1215; Bachem), cathepsin D (Cat. No. ES001; R&D Systems), cathepsin H (Cat. No. 05859; Chemimpex), and cathepsin S (Cat. No. ES002; R&D Systems). The peptide substrates are utilized to evaluate the cleaving efficiency of individual lysosomal isolation from different cell types. 5 μl of 200 μM peptide substrate is incubated with 5 μl of lysosomal extraction in citrate buffer (pH 5) for 30 min. at 37° C. Fluorescence emission of each peptide substrate was detected at specific wavelengths based on the fluorophore attached. Fluorescence level was normalized by subtracting the background fluoresce generated by the peptide substrate only in citrate buffer. Higher fluorescence signal detected indicates higher level of the enzymatic substrate cleaving activity of the particular cathepsin from the lysosome enrichment.

Engineering peptidase-cleavable substrates into GS1 and/or GS2 linker of a pIII: phage clones with cleavable substrate(s) are generated using wild type M13 bacteriophage vectors or recombinantly engineered variants thereof (see, e.g., Intl. Patent Application Publication No. WO 2017/091467, US Patent Application Publication No. 2018/0327480, and/or Afshar, S., et al., Protein Engineering, Design and Selection, 2020, vol. 33, pp. 1-8). Escherichia coli strain RZ1032 (Cat. No. 39737, ATCC), which lacks functional dUTPase and uracil glycosylase, is used to prepare uracil-containing ss DNA (du-ssDNA) of the M13 IX104 bacteriophage vector.

Oligonucleotide sequences encoding the five-residue FLVIR sequence (SEQ ID NO: 4) are designed, and the corresponding reverse complement oligo is annealed to various locations in pIII GS2 linker region of du-ssDNA IX104 vector by Kunkle mutagenesis. Electrocompetent E. coli DH10B cells (Cat. NO. 18290015, Invitrogen) are used for transformations. The pool of transformants are random-picked and sequenced to confirm the substrate presence and determine substrate location. Forty phage clones are amplified in the presence of freshly grown XL-1 blue cells (in-house) overnight on LB plates at 37° C. The next day, polymerase chain reaction (PCR) is performed to amplify the gene III sequence, which encodes pIII of each phage clone. PCR products are then sequenced to confirm the presence of corresponding cleavable substrate(s) in GS2 linker.

Ten rounds of overnight phage culture described above are grown to evaluate substrate sequence retention for each phage clone. Sanger sequencing is performed after each round of phage culture to confirm the substrate insertion. Final phage clones with the substrate insertion are then evaluated for cathepsin accessibility by incubation with lysosomal extract from different mammalian cell lines.

### Results:

To maximize the diminishing effect in phage infectivity, the FLVIR sequence (SEQ ID NO: 4) is inserted into GS2 linker of pIII to completely remove the N1 and N2 domains upon cathepsins digestion. The FLVIR sequence (SEQ ID NO: 4) is inserted randomly in the linker regions with single or multiple copies by Kunkle mutagenesis reactions resulting in 40 phage clones. To ensure the retention of the substrate sequence in place throughout multiple rounds of selection process, 10 rounds of overnight phage culture are completed with sequencing confirmation after each round. After 10 rounds of culture, 18 unique phage clones are harvested with either 1, 2 or 3 copies of FLVIR (SEQ ID NO: 4) inserted into the linker sites (Table 1). Grey boxes indicate the location of the inserted substrate sequence. Both GS1 and GS2 linkers are glycine (Gly)- and serine (Ser)-rich sequences with high similarity in nucleotide sequences. Therefore, a few of FLVIR sequences (SEQ ID NO: 4) occur in the GS1 linker in addition to GS2 linker.

The accessibility of the engineered GS2 linker of the 18 phage clones to active cathepsins (confirmed by fluorogenic cleaving assay) is assessed by incubating phage clones with CHO cell lysosomal extracts at 37° C. The assessment is repeated 4 times with independently isolated lysosome under acidic environment (about pH 5). Trends of and naked phage remains with fully infectious ability (as shown in the representative graph in FIG. 2). Among all phage clones, A1 and H4 are highly consistent, representing high, if not full, infectivity and low infectivity (9800 reduction), respectively (A1 clone, mean=114.8%, SEM=19.11%; H4 clone, mean=2%, SEM=0.7%; naked phage, mean=107%, SEM=5.9%, n=4)(data not shown). Both phage clones contain the same backbone as naked phage (NP), except the difference of engineered GS2 linkers, indicating that the engineered substrate in H4 clone is very effective.

The accessibility of clone A1 and H4 to active cathepsins is further assessed in lysosomal extracts from CaCo2 and HEK293 cells in addition to CHO cells. Although the fluorogenic cleaving assay suggests a slightly shifted cathepsin profiles in different cell types (Table 2), lysosomal cathepsins continued to recognize and cleave FLVIR sequences (i.e., SEQ ID NO: 4) that are engineered in clone H4 phage leading to a significant lower infectivity after 30 min. incubation at 37° C. (see, FIG. 3). CaCo2 lysosome: clone A1 (mean=79.83%, SEM=9.5%) vs. H4 (mean=34.63%, SEM=13.5%), p=0.22, n=3. HEK293 lysosome: clone A1 (mean=75.1%, SEM=9.7%) vs. H4 (mean=9.5%, SEM=1.6%), p=0.018, n=3. CHO lysosome: clone A1 (mean=95.58%, SEM=13.8%) vs. H4 (mean=2.738%, SEM=0.8%), p<0.0001, n=6.

TABLE 2
Activity evaluation of lysosomal isolation from different
cell types by cathepsin fluorogenic substrates

| | Lysosomal preparation | | | | | |
|---|---|---|---|---|---|---|
| | CaCo2 cells | | HEK cells | | CHO cells | |
| | Cleavage fluorescence intensity (normalized) | | | | | |
| | | 37° C. | | 37° C. | | 37° C. |
| | 0 min. | 30 min. | 0 min. | 30 min. | 0 min. | 30 min. |
| Cath A substrate | 74.076 | 1056.946 | 76.665 | 1608.554 | 216.232 | 4695.394 |
| Cath B/L | 95.067 | 5607.41 | 286.499 | 16573.77 | 676.577 | 23502.64 |
| substrate | | | | | | |
| Cath C substrate | 1317.411 | 16523.27 | 1917.683 | 27173.22 | 4.059 | 6.107 |
| Cath D substrate | 38.594 | 1453.774 | 38.22 | 2217.762 | 90.872 | 2438.224 |
| Cath H substrate | 0 | 174.262 | 0 | 114.905 | 0 | 56.29 |
| Cath S substrate | 17.079 | 270.806 | 113.167 | 2693.823 | 105.175 | 1982.617 |

### Example 2: Phage Display Library Construction and CPPs Selection

### Methods:

Cells and reagents: Neuro2a (N2a) cells are cultured in DMEM (Cat. No. 10-017-CV, Corning) supplemented with 10% HI FBS (Cat. No. 35-011-CV, Corning), in 5% CO₂ at 37° C. SH-SY5Y cells are grown in Eagle's minimal essential medium (EMEM) (Cat. No. MT10009CV, Corning) and Ham's F12 medium (Cat. No. 12-615F, Lonza) in a one-to-one ratio, supplemented with 10% HI FBS, in 5% CO₂ at 37° C. HEK, CaCo2 cells are maintained as previously described.

Phage display libraries: peptide phage libraries are generated based on the selected backbone structure of a desired M13 bacteriophage vector (for example, in the 8+11 vector based on the selected backbone structure of the IX104 bacteriophage vector) with the cathepsin-cleavable substrate insertion in GS2 linker.

A nine-residue library of oligonucleotides (9NNK) encoding random amino acid sequences is designed such that the random NNK region is flanked by nucleotides complementary to the vector. The 5′-phosphorylated reverse complement oligo is annealed to du-ss DNA 8+11 vector using Kunkel mutagenesis and extended to form dsDNA (Sidhu et al. (2000) Methods Enzymol. 328:333-363). Specifically, and based on the backbone structure of phage clone H4, a randomized peptide library is constructed with nine amino acids in length (i.e., 9NNK) displayed at the N-terminus of phage pIII. The diversity of the H4_9NNK library is approximately 7×10⁸ pfu.

Electrocompetent E. coli DH10B cells are used for transformations. A pool of transformants is titered to determine the diversity of the library. Phage are then amplified in the presence of freshly grown XL-1 blue cells overnight on LB plates at 37° C. The next day, phage is eluted off the plate, precipitated, titered and stored at −80° C. in the presence of 50% glycerol until use.

Before applying the engineered phage library to mammalian cells, complete culture medium is replaced with serum-free culture medium, and cells are incubated for 1 hr. at 37° C. For primary selection, 10¹² phage from the library are incubated with 10⁷ of various cultured cells as different selection arms for 1 hr. at 37° C. inside a tissue culture incubator (on rotator for suspension cells). This allows the internalization to occur leading by displayed peptides on phage. During internalization, if a phage particle displaying a particular peptide penetrates in cells and travels to lysosomes via cellular trafficking, the one or more FLVIR sequences (SEQ ID NO: 4) in phage pIII is accessed and cleaved by lysosomal cathepsins, which results in the loss of phage infectivity.

After internalization, cells are gently washed with cold phosphate buffer saline (PBS) once, and followed by cold, low-pH stripping buffer (culture medium is adjusted to pH 2.5 for CHO cells; 100 mM glycine, 150 mM NaCl, pH 2.5 for 293 and CaCo2 cells) for 5 min. twice. Then cells are immediately washed with cold PBS for 3 times. Cells in suspension are centrifuged at 300×g for 5 min. at 4° C., whereas adherent cells are scraped on ice and proceeded directly to the next step. Washed cells are gently lysed using the cytosolic extract reagents (ThermoFisher Scientific) to collect phage particles in about 1.5 ml volume. Phage from serum-free medium after internalization combined with the first PBS wash (considered as outside the cells) and cytosolic extracts (considered as inside the cells) are tittered separately to evaluate phage recovery compared to input. The recovered phage from the cytosolic region are amplified by plating with 5 mL of freshly grown mid-log XL-1 blue cells with 40 mL top agar onto large LB plates (Cat. No. L6100, Teknova). Plates are incubated overnight at 37° C. On the second day, the LB plates are first equilibrated to room temperature, and phage are eluted by incubation with 30 mL phage suspension buffer (100 mM NaCl, 8.1 mM MgCl₂, 50 mM Tris-HCl, pH 7.5) for 2 hr. at room temperature.

Then, the plate surface is gently scraped, and the phage elution is collected. The eluted phage samples are spined, precipitated and titered for use in subsequent rounds of selection. Five rounds of selection are conducted. Starting from the output of round (ORD) three to the completion of the whole selection, phage plaques are random-picked, eluted, PCR-amplified and sequenced by Sanger sequencing. The amplified phage samples of ORD 3-5, serving as the input rounds (TRD) 4-6, are analyzed by Next Generation Sequencing (NGS) to identify peptide sequences and their occurring frequencies.

Sanger sequencing: amplicons are first purified using Exonuclease I and Fast AP. The purified PCR product is used as the DNA template for the Big Dye Terminator 3.1 cycle sequencing chemistry. The sequencing reaction then is purified with Seq DTR MagBind beads and loaded onto a Bioanalyzer 3730XL for sequencing by capillary electrophoresis.

NGS: amplicons go through a 2-step PCR process. The first PCR step is adding the SBS sites for Illumina's sequencing primer, and the second PCR step is adding the Nextera Indexes to allow for sample demultiplexing. Both PCR steps are purified using a 1.8× ratio of MagBind RxnPurePlus beads. The purified PCR products are quantified by qPCR using a ViiA 7 and a Bioanalyzer 2100 fragment analyzer. The samples are then pooled in equal molar ratios and are denatured following Illumina's MiSeq System Denature and Dilute guide. Samples are loaded on a MiSeq at a concentration of 12.5 pM and 20% PhiX is spiked in. The run conditions for the MiSeq are a single direction of 130 cycles and 1 M reads via V2 Nano Reagent Kit.

Immunocytochemistry and confocal microscopy: cells are fixed with 4% paraformaldehyde (PFA) on ice for 20 min. after the washing steps. Fixed cells are washed with PBS for three times to remove any excess PFA and followed by 1 hr. blocking in 3% bovine serum albumin (BSA) in Tris buffer saline (TBS) with 0.2% Triton X-100 and antibodies staining in TBS with 0.2% triton X-100. Primary antibodies are incubated with cell samples overnight at 4° C. Following PBS washes, species-specific Alexa Fluor 488, Alexa Fluor 568 and/or Alexa Fluor 647-coupled secondary antibodies (Life Technologies; Grand Island, NY) are used for signal detection.

Imaging analysis is conducted on a confocal microscopy using a laser-scanning microscope 800 NLO (Zeiss) equipped with an argon laser. Primary antibodies are as follows: anti-M13-Alexa647 (in-house), anti-LAMP1, anti-F-actin-DyLight488 and DAPI. Controls treated with secondary antibody only show negative or undetectable signal.

Lipid interaction assessment by circular dichroism (CD): secondary structure characterization of CPPs is conducted in the presence of ultra-pure water and 1×HBS-N buffer. Similarly, CD spectra is collected in the presence of model lipid membrane (POPC) on the structural state of CPPs using JASCO-1500 CD spectrometer. 1 mg/mL of peptide is transferred into 0.02 cm path length quartz cuvette for far UV-CD measurement. POPC is added in equal concentration (w/v). All the measurements are done at room temperature (20° C.). Spectra is collected in 250-190 nm wavelength range. Furthermore, peptide spectra are corrected by subtracting with appropriate control. Secondary structure quantitative analysis is done using SSE multivariate analysis.

Far-UV CD parameters during spectrum measurement: CD spectrum measurement is conducted in standard mdeg mode. Scanning speed: 50 nm/min.; Response: 2 sec; Band width: 2 nm; [q]. Ellipticity is converted to mean residual Ellipticity using the following formula:

$${[ q ]}_{M R E} = q * MRW / cl ,$$

where MRW=(molecular weight of protein or peptide/number of peptide bonds in the protein.

Deconvolution of the circular dichroism spectra to calculate the % helix, beta sheet and random coil structure is conducted via CD multivariate SSE in JASCO-1500. CD instrument ID number: M610823.

Peptide synthesis and conjugation: synthetic peptides are ordered from CPC scientific with 90-95% purity. Chemical conjugation such as CPP to siRNA are conducted in-house. NNJA peptides in the formats of monomer or dendrimer are conjugated to siRNA targeting hypoxanthine-guanine phosphoribosyltransferase (HPRT) gene (designed in-house, synthesized from Biosynthesis) at the C-terminal end of the peptide by click chemistry.

NNJA-siRNA knock down assay: Ten thousand cells, such as HEK, N2a and SH-SY5Y cells are plated in Accell media followed by the treatment of the compounds (siRNA controls, NNJA-siRNA, or cholesterol-siRNA). The concentration of tested compounds starts at 2 μM followed by 1:5 dilutions. Cells are then incubated at 37° C., with 5% CO₂ for 72 hr. The knock-down efficiency achieved by the compounds (i.e., NNJA-siRNA) is assessed by qRT-PCR using Cells to Ct followed by TaqMan (Cat. No. A25603, ThermoFisher) with HPRT primer/probes. Cell viability is evaluated by CytoTox 96 Non-Radioactive Cytotoxicity Assay (Cat. No. G1780, Promega). Statistic analysis is generated in Prism using 3-parameter curve fit.

Statistics: statistical analysis is conducted using standard error of the mean (SEM), two-way ANOVA and multiple comparison test on GraphPad Prism (version 9.1.1), unless otherwise stated. Statistical results (e.g., p value) are described in figure legends and use confidence intervals of 95%.

### Results:

The output/input ratio of the phage titer recovery of each round of selection is increased as the selection rounds progressed (Table 3). The results indicate that the peptides that are displayed on phage having cell-permeability are enriched gradually by the cell-based panning process in each cell type. An examination of the occurrence of the 20 naturally occurring amino acids at each of the nine positions from the naïve library prior to the cell-based selection by Sanger sequencing of randomly picked plaques shows fairly equal frequency of amino acids are observed with no residue-bias at each position (data not shown).

The same analysis then is performed with enriched CPPs from later rounds of the selection. Peptide sequences identified from random-picked phage plaques from three selection arms are combined and summarized by their selection rounds—ORD3, 4 and 5 (data not shown). Overall, patterns begin to reveal after three rounds of selection, and specific residues are favored at particular positions. For example, methionine (Met) and leucine (Leu) are dominant at the first position (N-terminus), whereas, serine (Ser) and threonine (Thr) share the main frequency of the second position. Proline (Pro) repeats accumulate at the middle and the end of the peptide sequences. The biased pattern is also consistent when three selection arms from ORD5 are evaluated individually, yet with cell-type preferences (data not shown), such as at position 3, 4 and 6. Strikingly, all the CPPs discovered from the three selection arms are linear with very high isoelectric point (PI) values (majority PI are ˜9-12) (data not shown).

TABLE 3
Percentage of the phage titer recovery from cytosol domain after
each round of selection. Output phage titers are normalized
to input titer and shown as the percentage of recovery.

| Cell type | ORD1 | ORD2 | ORD3 | ORD4 | ORD5 |
|---|---|---|---|---|---|
| CHO | 0.038 | 0.003 | 0.012 | 0.048 | 0.6 |
| expi293 | 0.035 | 0.009 | 0.065 | 0.75 | 2.4 |
| CaCo2 | 0.00017 | 0.0015 | 0.006 | 0.012 | 0.018 |

To confirm internalization of the CPPs, the phage samples from IRD6 (amplified ORD5) are first tested for internalization in HEK cells by confocal microscopy. IRD6 phage from 3 selection arms, together with 2 negative controls (e.g., naked phage and naïve library phage) are added separately to adherent HEK cells and incubated for 1 hr. at 37° C. before processing for imaging. Penetrated phage particles are detected by ani-M13 antibody under confocal microscopy. Cell membrane is outlined by staining with filament actin antibody, and nucleus is probed by DAPL Minimal signal of anti-M13 antibody is detected from the control groups indicating neither naked phage nor naïve phage library penetrate HEK cells by themselves. In the enriched CPPs groups resulted from the selection, signal intensity of internalized phage particles is mainly detected in the cytosolic region and is significantly elevated. Compared to peptide-phage selected from CHO and CaCo2 cells, peptide-phage selected from 293 cells (IRD6_293) show higher internalization level in HEK cells indicating the cell-type preference.

In addition, subcellular localization of internalized phage particles from IRD6_293 pool in HEK cells is assessed under confocal microscopy with the z-stack function. Phage signals detected by anti-M13 antibody are located within the cytoplasmic region and are not co-localized with EEA1 (early endosome) or LAMP1 (lysosome) staining. The results suggest that IRD6_293 peptides appear to enter the cytoplasmic domain of HEK cells and further valid the mechanism of action of the novel phage library and selection process. Comparable results are observed with same phage samples internalized in CaCo2 cells. Increased phage signal is detected in the cytoplasmic domain and is not co-localized with EEA1 or LAMP1 staining.

Among the internalized peptides with high occurring frequency, 37 NNJA peptides are selected with the most occurrence and/or enrichment from the three selection arms based on NGS analysis and constructed as homogenous (monoclonal) NNJA-phage samples (i.e., NNJA peptides). Some NNJA peptides sequences are shared from the 3 cell selection arms, while the others are cell-type preferential or specific (Table 4) indicating that distinguished internalization mechanisms of the peptides are utilized in different cell types. Penetration and subcellular localization of purified peptide-phage is assessed in HEK and CaCo2 cells by Confocal imaging. Homogenous NNJA peptides on phage are added to cells for 1 hr. internalization at 37° C. Cells are washed and surface bound phage are stripped sufficiently followed by immunocytochemistry staining and Confocal microscopy imaging. Different levels of cytosolic internalization with NNJA peptides on phage are summarized in Table 4. The internalization levels of NNJA peptides in HEK and CaCo2 cells are generally consistent with the level of the occurrence from the cell-type selections respectively, based on NGS analysis (data not shown).

TABLE 4
Putative CPP Amino Acid Sequences and
Cell Type Cytosolic Internalization.

Cell Type internalization
detected by Confocal
microscopy

| Peptide | HEK cells | CaCo2 cells |
|---|---|---|
| NNJA_1 (SEQ ID NO: 12) | +++ | ++ |
| NNJA_2 (SEQ ID NO: 13) | + | +/− |
| NNJA_3 (SEQ ID NO: 14) | + | +/− |
| NNJA_4 (SEQ ID NO: 15) | ++ | +/− |
| NNJA_5 (SEQ ID NO: 16) | ++ | ++ |
| NNJA_6 (SEQ ID NO: 17) | ++ | +/− |
| NNJA_7 (SEQ ID NO: 18) | ++ | +++ |
| NNJA_8 (SEQ ID NO: 19) | + | +/− |
| NNJA_9 (SEQ ID NO: 20) | + | +/− |
| NNJA_10 (SEQ ID NO: 21) | + | +/− |
| NNJA_11 (SEQ ID NO: 22) | +++ | ++ |
| NNJA_12 (SEQ ID NO: 23) | ++ | +/− |
| NNJA_13 (SEQ ID NO: 24) | +++ | +/− |
| NNJA_14 (SEQ ID NO: 25) | + | +/− |
| NNJA_15 (SEQ ID NO: 26) | +++ | +++ |
| NNJA_16 (SEQ ID NO: 27) | ++ | +/− |
| NNJA_17 (SEQ ID NO: 28) | +++ | +/− |
| NNJA_18 (SEQ ID NO: 29) | ++ | +/− |
| NNJA_19 (SEQ ID NO: 30) | ++ | + |
| NNJA_20 (SEQ ID NO: 31) | ++ | + |
| NNJA_21 (SEQ ID NO: 32) | + | + |
| NNJA_22 (SEQ ID NO: 33) | + | +/− |
| NNJA_23 (SEQ ID NO: 34) | ++ | − |
| NNJA_24 (SEQ ID NO: 35) | + | − |
| NNJA_25 (SEQ ID NO: 36) | + | +/− |
| NNJA_26 (SEQ ID NO: 37) | + | +/− |
| NNJA_27 (SEQ ID NO: 38) | + | + |
| NNJA_28 (SEQ ID NO: 39) | + | +++ |
| NNJA_29 (SEQ ID NO: 40) | +++ | +/− |
| NNJA_30 (SEQ ID NO: 41) | +++ | +++ |
| NNJA_31 (SEQ ID NO: 42) | + | +/− |
| NNJA_32 (SEQ ID NO: 43) | + | +/− |
| NNJA_33 (SEQ ID NO: 44) | + | +/− |
| NNJA_34 (SEQ ID NO: 45) | +/− | + |
| NNJA_35 (SEQ ID NO: 46) | ++ | ++ |
| NNJA_36 (SEQ ID NO: 47) | +/− | +/− |
| NNJA_37 (SEQ ID NO: 48) | +/− | +/− |

Peptide NNJA_15 on phage is further evaluated by Confocal microscopy in additional cell types to assess penetration, including N2a and SH-SY5Y cells. Phage sample is introduced to the targeted cells and allowed internalization for 1 hr. at 37° C. Cells are then processed as describe previously for Confocal imaging and analysis. NNJA-15 on phage is detected at a modest level by anti-M13 antibody in the cytoplasmic domain with no co-localization with LAMP1 staining, in both N2a and SH-SY5Y cells. The results suggest that NNJA peptides may penetrate in cell types in addition to the ones they are screened against initially.

To assess if NNJA peptides as synthetic peptides can further delivery cargos in mammalian cells, selected peptides are conjugated to siRNA targeting HPRT gene for self-delivery assessment. In addition to the monomer format of NNJA peptides, dendrimeric peptides which mimicking the multi-copy and structure of peptides displayed on phage are evaluated. The compounds are introduced to various cell types (e.g. HEK, N2a and SH-SY5Y cells), and the knockdown efficiency of HPRT gene is investigated shown in the percentage of RNA remaining after 72 hr. (see, FIG. 4A). HPRT siRNA conjugated to cholesterol serves as the positive control, and naked siRNA and non-targeting control (NTC) siRNA-cholesterol serve as the negative controls. Overall, NNJA dendrimers provide increased penetration level leading to higher siRNA knockdown, and a few of the tested dendrimers achieve about 80% gene reduction, with a single digit nanomolar level of the half-maximal inhibitory concentration (IC₅₀) value (not shown). The results suggest that multivalency of the peptides help with the penetration rate. Interestingly, the monomeric format of NNJA_1 facilitate the siRNA entry and achieve higher knockdown in HEK and N2a cells compared to their dendrimers, whereas the dendrimers behave better in SH-SY5Y cells. NNJA_5 monomer provide superior penetration compared to their dendrimer counterpart for the siRNA delivery in all three cell types.

The cell viability measured by lactate dehydrogenase (LDH) release is shown in FIG. 4B. NNJA peptides do not induce significant cell death compared to the controls. In N2a cells, a lower viability is observed in the NNJA dendrimer group; however, the viability is recovered under a higher treatment concentration of the peptide-siRNA. The viability indicated by the LDH release may not reflect real cytotoxicity, but a temperate LDH release under certain treatment conditions.

To study the potential mechanisms of action of NNJA peptides, 4 of the highly internalized NNJA peptides are evaluated as synthetic monomer peptides by circular dichroism (CD) spectroscopy in the presence of liposome for potential lipid interaction. 1-Palmitoyl-2-oleoyl-sn-glycero-3-phosphocholine (POPC) is the one of the most common liposomes representing lipid components of mammalian cell plasma membranes and is used in this assay for biophysical evaluation. All 4 peptides presented similar secondary structure signature, yet differed in the secondary structure content (e.g., helix, sheet and turn (data not shown)). Upon interacting with POPC, a significant change (shown in dash line) in the intensity and CD signal maximum is observed in the secondary structure signature of NNJA_19, but not NNJA_1, NNJA_5 or NNJA_15. As such, it appears that direct interaction with the lipid bilayer may be the penetration mechanism for NNJA_19, whereas NNJA_1, NNJA_5 or NNJA_15 appear to utilize different mechanisms to enter cytoplasmic domain, such as endocytosis pathway (see, FIG. 5).

### Listing of Sequences

The following nucleic and/or amino acid sequences are referred to in the disclosure and are provided below for reference.

Wild-Type M13 Nucleic Acid Sequence
SEQ ID NO: 1
aacgctactactattagtagaattgatgccaccttttcagctcgcgccccaaatgaaaatatagctaaacaggttattgaccatttgc
gaaatgtatctaatggtcaaactaaatctactcgttcgcagaattgggaatcaactgttacatggaatgaaacttccagacaccgtac
tttagttgcatatttaaaacatgttgagctacagcaccagattcagcaattaagctctaagccatccgcaaaaatgacctcttatcaa
aaggagcaattaaaggtactctctaatcctgacctgttggagtttgcttccggtctggttcgctttgaagctcgaattaaaacgcgat
atttgaagtctttcgggcttcctcttaatctttttgatgcaatccgctttgcttctgactataatagtcagggtaaagacctgatttt
tgatttatggtcattctcgttttctgaactgtttaaagcatttgagggggattcaatgaatatttatgacgattccgcagtattggac
gctatccagtctaaacattttactattaccccctctggcaaaacttcttttgcaaaagcctctcgctattttggtttttatcgtcgtc
tggtaaacgagggttatgatagtgttgctcttactatgcctcgtaattccttttggcgttatgtatctgcattagttgaatgtggtat
tcctaaatctcaactgatgaatctttctacctgtaataatgttgttccgttagttcgttttattaacgtagatttttcttcccaacgt
cctgactggtataatgagccagttcttaaaatcgcataaggtaattcacaatgattaaagttgaaattaaaccatctcaagcccaatt
tactactcgttctggtgtttctcgtcagggcaagccttattcactgaatgagcagctttgttacgttgatttgggtaatgaatatccg
gttcttgtcaagattactcttgatgaaggtcagccagcctatgcgcctggtctgtacaccgttcatctgtcctctttcaaagttggtc
agttcggttcccttatgattgaccgtctgcgcctcgttccggctaagtaacatggagcaggtcgcggatttcgacacaatttatcagg
cgatgatacaaatctccgttgtactttgtttcgcgcttggtataatcgctgggggtcaaagatgagtgttttagtgtattctttcgcc
tctttcgttttaggttggtgccttcgtagtggcattacgtattttacccgtttaatggaaacttcctcatgaaaaagtctttagtcct
caaagcctctgtagccgttgctaccctcgttccgatgctgtctttcgctgctgagggtgacgatcccgcaaaagcggcctttaactcc
ctgcaagcctcagcgaccgaatatatcggttatgcgtgggcgatggttgttgtcattgtcggcgcaactatcggtatcaagctgttta
agaaattcacctcgaaagcaagctgataaaccgatacaattaaaggctccttttggagcctttttttttggagattttcaacatgaaa
aaattattattcgcaattcctttagttgttcctttctattctcactccgctgaaactgttgaaagttgtttagcaaaaccccatacag
aaaattcatttactaacgtctggaaagacgacaaaactttagatcgttacgctaactatgagggttgtctgtggaatgctacaggcgt
tgtagtttgtactggtgacgaaactcagtgttacggtacatgggttcctattgggcttgctatccctgaaaatgagggtggtggctct
gagggtggcggttctgagggtggcggttctgagggtggcggtactaaacctcctgagtacggtgatacacctattccgggctatactt
atatcaaccctctegacggcacttatccgcctggtactgagcaaaaccccgctaatcctaatccttctcttgaggagtctcagcctct
taatactttcatgtttcagaataataggttccgaaataggcagggggcattaactgtttatacgggcactgttactcaaggcactgac
cccgttaaaacttattaccagtacactcctgtatcatcaaaagccatgtatgacgcttactggaacggtaaattcagagactgcgctt
tccattctggctttaatgaggatccattcgtttgtgaatatcaaggccaatcgtctgacctgcctcaacctcctgtcaatgctggcgg
cggctctggtggtggttctggtggcggctctgagggtggtggctctgagggtggcggttctgagggtggcggctctgagggaggcggt
tccggtggtggctctggttccggtgattttgattatgaaaagatggcaaacgctaataagggggctatgaccgaaaatgccgatgaaa
acgcgctacagtctgacgctaaaggcaaacttgattctgtcgctactgattacggtgctgctatcgatggtttcattggtgacgtttc
cggccttgctaatggtaatggtgctactggtgattttgctggctctaattcccaaatggctcaagtcggtgacggtgataattcacct
ttaatgaataatttccgtcaatatttaccttccctccctcaatcggttgaatgtcgcccttttgtctttagcgctggtaaaccatatg
aattttctattgattgtgacaaaataaacttattccgtggtgtctttgcgtttcttttatatgttgccacctttatgtatgtattttc
tacgtttgctaacatactgcgtaataaggagtcttaatcatgccagttcttttgggtattccgttattattgcgtttcctcggtttcc
ttctggtaactttgttcggctatctgcttacttttcttaaaaagggcttcggtaagatagctattgctatttcattgtttcttgctct
tattattgggcttaactcaattcttgtgggttatctctctgatattagcgctcaattaccctctgactttgttcagggtgttcagtta
attctcccgtctaatgcgcttccctgtttttatgttattctctctgtaaaggctgctattttcatttttgacgttaaacaaaaaatcg
tttcttatttggattgggataaataatatggctgtttattttgtaactggcaaattaggctctggaaagacgctcgttagcgttggta
agattcaggataaaattgtagctgggtgcaaaatagcaactaatcttgatttaaggcttcaaaacctcccgcaagtcgggaggttcgc
taaaacgcctegcgttcttagaataccggataagccttctatatctgatttgcttgctattgggcgcggtaatgattcctacgatgaa
aataaaaacggcttgcttgttctcgatgagtgcggtacttggtttaatacccgttcttggaatgataaggaaagacagccgattattg
attggtttctacatgctcgtaaattaggatgggatattatttttcttgttcaggacttatctattgttgataaacaggcgcgttctgc
attagctgaacatgttgtttattgtcgtcgtctggacagaattactttaccttttgtcggtactttatattctcttattactggctcg
aaaatgcctctgcctaaattacatgttggcgttgttaaatatggcgattctcaattaagccctactgttgagcgttggctttatactg
gtaagaatttgtataacgcatatgatactaaacaggctttttctagtaattatgattccggtgtttattcttatttaacgccttattt
atcacacggtcggtatttcaaaccattaaatttaggtcagaagatgaaattaactaaaatatatttgaaaaagttttctcgcgttctt
tgtcttgcgattggatttgcatcagcatttacatatagttatataacccaacctaagccggaggttaaaaaggtagtctctcagacct
atgattttgataaattcactattgactcttctcagcgtcttaatctaagctatcgctatgttttcaaggattctaagggaaaattaat
taatagcgacgatttacagaagcaaggttattcactcacatatattgatttatgtactgtttccattaaaaaaggtaattcaaatgaa
attgttaaatgtaattaattttgttttcttgatgtttgtttcatcatcttcttttgctcaggtaattgaaatgaataattcgcctctg
cgcgattttgtaacttggtattcaaagcaatcaggcgaatccgttattgtttctcccgatgtaaaaggtactgttactgtatattcat
ctgacgttaaacctgaaaatctacgcaatttctttatttctgttttacgtgctaataattttgatatggttggttcaattccttccat
aattcagaagtataatccaaacaatcaggattatattgatgaattgccatcatctgataatcaggaatatgatgataattccgctcct
tctggtggtttctttgttccgcaaaatgataatgttactcaaacttttaaaattaataacgttcgggcaaaggatttaatacgagttg
tcgaattgtttgtaaagtctaatacttctaaatcctcaaatgtattatctattgacggctctaatctattagttgttagtgcacctaa
agatattttagataaccttcctcaattcctttctactgttgatttgccaactgaccagatattgattgagggtttgatatttgaggtt
cagcaaggtgatgctttagatttttcatttgctgctggctctcagcgtggcactgttgcaggcggtgttaatactgaccgcctcacct
ctgttttatcttctgctggtggttcgttcggtatttttaatggcgatgttttagggctatcagttcgcgcattaaagactaatagcca
ttcaaaaatattgtctgtgccacgtattcttacgctttcaggtcagaagggttctatctctgttggccagaatgtcccttttattact
ggtcgtgtgactggtgaatctgccaatgtaaataatccatttcagacgattgagcgtcaaaatgtaggtatttccatgagcgtttttc
ctgttgcaatggctggcggtaatattgttctggatattaccagcaaggccgatagtttgagttcttctactcaggcaagtgatgttat
tactaatcaaagaagtattgctacaacggttaatttgcgtgatggacagactcttttactcggtggcctcactgattataaaaacact
tctcaagattctggcgtaccgttcctgtctaaaatccctttaateggcctcctgtttagctcccgctctgattccaacgaggaaagca
cgttatacgtgctcgtcaaagcaaccatagtacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagc
tctaaatcgggggctccctttagggttccgatttagtgctttacggcacctcgaccccaaaaaacttgatttgggtgatggttcacgt
agtgggccatcgccctgatagacggtttttcgccctttgacgttggagtccacgttctttaatagtggactcttgttccaaactggaa
caacactcaaccctatctcgggctattcttttgatttataagggattttgccgatttcggcctattggttaaaaaatgagctgattta
acaaaaatttaacgcgaattttaacaaaatattaacgtttacaatttaaatatttgcttatacaatcttcctgtttttggggcttttc
tgattatcaaccggggtacatatgattgacatgctagttttacgattaccgttcatcgattctcttgtttgctccagactctcaggca
atgacctgatagcctttgtagacctctcaaaaatagctaccctctccggcatgaatttatcagctagaacggttgaatatcatattga
tggtgatttgactgtctccggcctttctcacccttttgaatctttacctacacattactcaggcattgcatttaaaatatatgagggt
tctaaaaatttttatccttgcgttgaaataaaggcttctcccgcaaaagtattacagggtcataatgtttttggtacaaccgatttag
ctttatgctctgaggctttattgcttaattttgctaattctttgccttgcctgtatgatttattggatgtt
M13 IX104 Nucleic Acid Sequence
SEQ ID NO: 2
aatgctactactattagtagaattgatgccaccttttcagctegcgccccaaatgaaaatatagctaaacaggttattgaccatttgc
gaaatgtatctaatggtcaaactaaatctactcgttcgcagaattgggaatcaactgttacatggaatgaaacttccagacaccgtac
tttagttgcatatttaaaacatgttgagctacagcaccagattcagcaattaagctctaagccatctgcaaaaatgacctcttatcaa
aaggagcaattaaaggtactctctaatcctgacctgttggagtttgcttccggtctggttcgctttgaagctcgaattaaaacgcgat
atttgaagtctttcgggcttcctcttaatctttttgatgcaatccgctttgcttctgactataatagtcagggtaaagacctgatttt
tgatttatggtcattctcgttttctgaactgtttaaagcatttgagggggattcaatgaatatttatgacgattccgcagtattggac
gctatccagtctaaacattttactattaccccctctggcaaaacttcttttgcaaaagcctctcgctattttggtttttatcgtcgtc
tggtaaacgagggttatgatagtgttgctcttactatgcctcgtaattccttttggcgttatgtatctgcattagttgaatgtggtat
tcctaaatctcaactgatgaatctttctacctgtaataatgttgttccgttagttcgttttattaacgtagatttttcttcccaacgt
cctgactggtataatgagccagttcttaaaatcgcataaggtaattcacaatgattaaagttgaaattaaaccatctcaagcccaatt
tactactcgttctggtgtttctcgtcagggcaagccttattcactgaatgagcagctttgttacgttgatttgggtaatgaatatccg
gttcttgtcaagattactcttgatgaaggtcagccagcctatgcgcctggtctgtacaccgttcatctgtcctctttcaaagttggtc
agttcggttcccttatgattgaccgtctgcgcctcgttccggctaagtaacatggagcaggtcgcggatttcgacacaatttatcagg
cgatgatacaaatctccgttgtactttgtttcgcgcttggtataatcgctgggggtcaaagatgagtgttttagtgtattctttcgcc
tctttcgttttaggttggtgccttcgtagtggcattacgtattttacccgtttaatggaaacttcctcatgaaaaagtctttagtcct
caaagcctctgtagccgttgctaccctcgttccgatgctgtctttcgctgctgagggtgacgatcccgcaaaagcggcctttaactcc
ctgcaagcctcagcgaccgaatatatcggttatgcgtgggcgatggttgttgtcattgtcggcgcaactatcggtatcaagctgttta
agaaattcacctcgaaagcaagctgataaaccgatacaattaaaggctccttttggagcctttttttttggagattttcaacgtgaaa
aaattattattcgcaattcctttagttgttcctttctattctcactccgctgaaactgttgaaagttgtttagcaaaatcccatacag
aaaattcatttactaacgtctggaaagacgacaaaactttagatcgttacgctaactatgagggctgtctgtggaatgctacaggcgt
tgtagtttgtactggtgacgaaactcagtgttacggtacatgggttcctattgggcttgctatccctgaaaatgagggtggtggctct
gagggtggcggttctgagggtggcggttctgagggtggcggtactaaacctcctgagtacggtgatacacctattccgggctatactt
atatcaaccctctcgacggcacttatccgcctggtactgagcaaaaccccgctaatcctaatccttctcttgaggagtctcagcctct
taatactttcatgtttcagaataataggttccgaaataggcagggggcattaactgtttatacgggcactgttactcaaggcactgac
cccgttaaaacttattaccagtacactcctgtatcatcaaaagccatgtatgacgcttactggaacggtaaattcagagactgcgctt
tccattctggctttaatgaggatttatttgtttgtgaatatcaaggccaatcgtctgacctgcctcaacctcctgtcaatgctggcgg
cggctctggtggtggttctggtggcggctctgagggtggtggctctgagggtggcggttctgagggtggcggctctgagggaggcggt
tccggtggtggctctggttccggtgattttgattatgaaaagatggcaaacgctaataagggggctatgaccgaaaatgccgatgaaa
acgcgctacagtctgacgctaaaggcaaacttgattctgtcgctactgattacggtgctgctatcgatggtttcattggtgacgtttc
cggccttgctaatggtaatggtgctactggtgattttgctggctctaattcccaaatggctcaagtcggtgacggtgataattcacct
ttaatgaataatttccgtcaatatttaccttccctccctcaatcggttgaatgtcgcccttttgtctttagcgctggtaaaccatatg
aattttctattgattgtgacaaaataaacttattccgtggtgtctttgcgtttcttttatatgttgccacctttatgtatgtattttc
tacgtttgctaacatactgcgtaataaggagtcttaatcatgccagttcttttgggtattccgttattattgcgtttcctcggtttcc
ttctggtaactttgttcggctatctgcttacttttcttaaaaagggcttcggtaagatagctattgctatttcattgtttcttgctct
tattattgggcttaactcaattcttgtgggttatctctctgatattagcgctcaattaccctctgactttgttcagggtgttcagtta
attctcccgtctaatgcgcttccctgtttttatgttattctctctgtaaaggctgctattttcatttttgacgttaaacaaaaaatcg
tttcttatttggattgggataaataatatggctgtttattttgtaactggcaaattaggctctggaaagacgctcgttagcgttggta
agattcaggataaaattgtagctgggtgcaaaatagcaactaatcttgatttaaggcttcaaaacctcccgcaagtcgggaggttcgc
taaaacgcctcgcgttcttagaataccggataagccttctatatctgatttgcttgctattgggcgcggtaatgattcctacgatgaa
aataaaaacggcttgcttgttctcgatgagtgcggtacttggtttaatacccgttcttggaatgataaggaaagacagccgattattg
attggtttctacatgctcgtaaattaggatgggatattatttttcttgttcaggacttatctattgttgataaacaggcgcgttctgc
attagctgaacatgttgtttattgtcgtcgtctggacagaattactttaccttttgtcggtactttatattctcttattactggctcg
aaaatgcctctgcctaaattacatgttggcgttgttaaatatggcgattctcaattaagccctactgttgagcgttggctttatactg
gtaagaatttgtataacgcatatgatactaaacaggctttttctagtaattatgattccggtgtttattcttatttaacgccttattt
atcacacggtcggtatttcaaaccattaaatttaggtcagaagatgaagcttactaaaatatatttgaaaaagttttcacgcgttctt
tgtcttgcgattggatttgcatcagcatttacatatagttatataacccaacctaagccggaggttaaaaaggtagtctctcagacct
atgattttgataaattcactattgactcttctcagcgtcttaatctaagctatcgctatgttttcaaggattctaagggaaaattaat
taatagcgacgatttacagaagcaaggttattcactcacatatattgatttatgtactgtttccattaaaaaaggtaattcaaatgaa
attgttaaatgtaattaattttgttttcttgatgtttgtttcatcatcttcttttgctcaggtaattgaaatgaataattcgcctctg
cgcgattttgtaacttggtattcaaagcaatcaggcgaatccgttattgtttctcccgatgtaaaaggtactgttactgtatattcat
ctgacgttaaacctgaaaatctacgcaatttctttatttctgttttacgtgctaataattttgatatggttggttcaattccttccat
aattcagaagtataatccaaacaatcaggattatattgatgaattgccatcatctgataatcaggaatatgatgataattccgctcct
tctggtggtttctttgttccgcaaaatgataatgttactcaaacttttaaaattaataacgttcgggcaaaggatttaatacgagttg
tcgaattgtttgtaaagtctaatacttctaaatcctcaaatgtattatctattgacggctctaatctattagttgttagtgcacctaa
agatattttagataaccttcctcaattcctttctactgttgatttgccaactgaccagatattgattgagggtttgatatttgaggtt
cagcaaggtgatgctttagatttttcatttgctgctggctctcagcgtggcactgttgcaggcggtgttaatactgaccgcctcacct
ctgttttatcttctgctggtggttcgttcggtatttttaatggcgatgttttagggctatcagttcgcgcattaaagactaatagcca
ttcaaaaatattgtctgtgccacgtattcttacgctttcaggtcagaagggttctatctctgttggccagaatgtcccttttattact
ggtcgtgtgactggtgaatctgccaatgtaaataatccatttcagacgattgagcgtcaaaatgtaggtatttccatgagcgtttttc
ctgttgcaatggctggcggtaatattgttctggatattaccagcaaggccgatagtttgagttcttctactcaggcaagtgatgttat
tactaatcaaagaagtattgctacaacggttaatttgcgtgatggacagactcttttactcggtggcctcactgattataaaaacact
tctcaagattctggcgtaccgttcctgtctaaaatccctttaatcggcctcctgtttagctcccgctctgattccaacgaggaaagca
cgttatacgtgctcgtcaaagcaaccatagtacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagc
tctaaatcgggggctccctttagggttccgatttagtgctttacggcacctcgaccccaaaaaacttgatttgggtgatggttcacgt
agtgggccatcgccctgatagacggtttttcgccctttgacgttggagtccacgttctttaatagtggactcttgttccaaactggaa
caacactcaaccctatctcgggctattcttttgatttataagggattttgccgatttcggtaaaaaatgagctgatttaacaaaaatt
taatgcgaattttaacaaaatattaacgtttacaatttaaatatttgcttatacaatcttcctgtttttggggcttttctgattatca
accggggtacatatgattgacatgctagttttacgattaccgttcatcgattctcttgtttgctccagactctcaggcaatgacctga
tagcctttgtagatctctcaaaaatagctaccctctccggcattaatttatcagctagaacggttgaatatcatattgatggtgattt
gactgtctccggcctttctcacccttttgaatctttacctacacattactcaggcattgcatttaaaatatatgagggttctaaaaat
ttttatccttgcgttgaaataaaggcttctcccgcaaaagtattacagggtcataatgtttttggtacaaccgatttagctttatgct
ctgaggctttattgcttaattttgctaattctttgccttgcctgtatgatttattggacgtt
Engineered M13 IX104 Nucleic Acid Sequence
SEQ ID NO: 3
aatgctactactattagtagaattgatgccaccttttcagctegcgccccaaatgaaaatatagctaaacaggttattgaccatttgc
gaaatgtatctaatggtcaaactaaatctactcgttcgcagaattgggaatcaactgttacatggaatgaaacttccagacaccgtac
tttagttgcatatttaaaacatgttgagctacagcaccagattcagcaattaagctctaagccatctgcaaaaatgacctcttatcaa
aaggagcaattaaaggtactctctaatcctgacctgttggagtttgcttccggtctggttcgctttgaagctcgaattaaaacgcgat
atttgaagtctttcgggcttcctcttaatctttttgatgcaatccgctttgcttctgactataatagtcagggtaaagacctgatttt
tgatttatggtcattctcgttttctgaactgtttaaagcatttgagggggattcaatgaatatttatgacgattccgcagtattggac
gctatccagtctaaacattttactattaccccctctggcaaaacttcttttgcaaaagcctctcgctattttggtttttatcgtcgtc
tggtaaacgagggttatgatagtgttgctcttactatgcctcgtaattccttttggcgttatgtatctgcattagttgaatgtggtat
tcctaaatctcaactgatgaatctttctacctgtaataatgttgttccgttagttcgttttattaacgtagatttttcttcccaacgt
cctgactggtataatgagccagttcttaaaatcgcataaggtaattcacaatgattaaagttgaaattaaaccatctcaagcccaatt
tactactcgttctggtgtttctcgtcagggcaagccttattcactgaatgagcagctttgttacgttgatttgggtaatgaatatccg
gttcttgtcaagattactcttgatgaaggtcagccagcctatgcgcctggtctgtacaccgttcatctgtcctctttcaaagttggtc
agttcggttcccttatgattgaccgtctgcgcctcgttccggctaagtaacatggagcaggtcgcggatttcgacacaatttatcagg
cgatgatacaaatctccgttgtactttgtttcgcgcttggtataatcgctgggggtcaaagatgagtgttttagtgtattctttcgcc
tctttcgttttaggttggtgccttcgtagtggcattacgtattttacccgtttaatggaaacttcctcatgaaaaagtctttagtcct
caaagcctctgtagccgttgctaccctcgttccgatgctgtctttcgctgctgagggtgacgatcccgcaaaagcggcctttaactcc
ctgcaagcctcagcgaccgaatatateggttatgcgtgggcgatggttgttgtcattgtcggcgcaactatcggtatcaagctgttta
agaaattcacctcgaaagcaagctgataaaccgatacaattaaaggctccttttggagcctttttttttggagattttcaacgtgaaa
aaattattattcgcaattcctttagttgttcctttctattctcactccgctgaaactgttgaaagttgtttagcaaaatcccatacag
aaaattcatttactaacgtctggaaagacgacaaaactttagatcgttacgctaactatgagggctgtctgtggaatgctacaggcgt
tgtagtttgtactggtgacgaaactcagtgttacggtacatgggttcctattgggcttgctatccctgaaaatgagggtggtggctct
gagggtggcggttctgagggtggcggttctgagggtggcggtactaaacctcctgagtacggtgatacacctattccgggctatactt
atatcaaccctctcgacggcacttatccgcctggtactgagcaaaaccccgctaatcctaatccttctcttgaggagtctcagcctct
taatactttcatgtttcagaataataggttccgaaataggcagggggcattaactgtttatacgggcactgttactcaaggcactgac
cccgttaaaacttattaccagtacactcctgtatcatcaaaagccatgtatgacgcttactggaacggtaaattcagagactgcgctt
tccattctggctttaatgaggatttatttgtttgtgaatatcaaggccaatcgtctgacctgcctcaacctcctgtcaatgctggcgg
cggctcttttttagttattagaggtggtggttctggtggcggctctgagggtggtggctctgagtttttagttattagaggtggcggt
tctgagggtggcggctctgagggaggcggttccggtggtggctctggttccggtgattttgattatgaaaagatggcaaacgctaata
agggggctatgaccgaaaatgccgatgaaaacgcgctacagtctgacgctaaaggcaaacttgattctgtcgctactgattacggtgc
tgctatcgatggtttcattggtgacgtttccggccttgctaatggtaatggtgctactggtgattttgctggctctaattcccaaatg
gctcaagtcggtgacggtgataattcacctttaatgaataatttccgtcaatatttaccttccctccctcaatcggttgaatgtcgcc
cttttgtctttagcgctggtaaaccatatgaattttctattgattgtgacaaaataaacttattccgtggtgtctttgcgtttctttt
atatgttgccacctttatgtatgtattttctacgtttgctaacatactgcgtaataaggagtcttaatcatgccagttcttttgggta
ttccgttattattgcgtttcctcggtttccttctggtaactttgttcggctatctgcttacttttcttaaaaagggcttcggtaagat
agctattgctatttcattgtttcttgctcttattattgggcttaactcaattcttgtgggttatctctctgatattagcgctcaatta
ccctctgactttgttcagggtgttcagttaattctcccgtctaatgcgcttccctgtttttatgttattctctctgtaaaggctgcta
ttttcatttttgacgttaaacaaaaaatcgtttcttatttggattgggataaataatatggctgtttattttgtaactggcaaattag
gctctggaaagacgctcgttagcgttggtaagattcaggataaaattgtagctgggtgcaaaatagcaactaatcttgatttaaggct
tcaaaacctcccgcaagtegggaggttcgctaaaacgcctcgcgttcttagaataccggataagccttctatatctgatttgcttgct
attgggcgcggtaatgattcctacgatgaaaataaaaacggcttgcttgttctcgatgagtgcggtacttggtttaatacccgttctt
ggaatgataaggaaagacagccgattattgattggtttctacatgctcgtaaattaggatgggatattatttttcttgttcaggactt
atctattgttgataaacaggcgcgttctgcattagctgaacatgttgtttattgtcgtcgtctggacagaattactttaccttttgtc
ggtactttatattctcttattactggctcgaaaatgcctctgcctaaattacatgttggcgttgttaaatatggcgattctcaattaa
gccctactgttgagcgttggctttatactggtaagaatttgtataacgcatatgatactaaacaggctttttctagtaattatgattc
cggtgtttattcttatttaacgccttatttatcacacggtcggtatttcaaaccattaaatttaggtcagaagatgaagcttactaaa
atatatttgaaaaagttttcacgcgttctttgtcttgcgattggatttgcatcagcatttacatatagttatataacccaacctaagc
cggaggttaaaaaggtagtctctcagacctatgattttgataaattcactattgactcttctcagcgtcttaatctaagctatcgcta
tgttttcaaggattctaagggaaaattaattaatagcgacgatttacagaagcaaggttattcactcacatatattgatttatgtact
gtttccattaaaaaaggtaattcaaatgaaattgttaaatgtaattaattttgttttcttgatgtttgtttcatcatcttcttttgct
caggtaattgaaatgaataattcgcctctgcgcgattttgtaacttggtattcaaagcaatcaggcgaatccgttattgtttctcccg
atgtaaaaggtactgttactgtatattcatctgacgttaaacctgaaaatctacgcaatttctttatttctgttttacgtgctaataa
ttttgatatggttggttcaattccttccataattcagaagtataatccaaacaatcaggattatattgatgaattgccatcatctgat
aatcaggaatatgatgataattccgctccttctggtggtttctttgttccgcaaaatgataatgttactcaaacttttaaaattaata
acgttcgggcaaaggatttaatacgagttgtcgaattgtttgtaaagtctaatacttctaaatcctcaaatgtattatctattgacgg
ctctaatctattagttgttagtgcacctaaagatattttagataaccttcctcaattcctttctactgttgatttgccaactgaccag
atattgattgagggtttgatatttgaggttcagcaaggtgatgctttagatttttcatttgctgctggctctcagcgtggcactgttg
caggcggtgttaatactgaccgcctcacctctgttttatcttctgctggtggttcgttcggtatttttaatggcgatgttttagggct
atcagttcgcgcattaaagactaatagccattcaaaaatattgtctgtgccacgtattcttacgctttcaggtcagaagggttctatc
tctgttggccagaatgtcccttttattactggtcgtgtgactggtgaatctgccaatgtaaataatccatttcagacgattgagcgtc
aaaatgtaggtatttccatgagcgtttttcctgttgcaatggctggcggtaatattgttctggatattaccagcaaggccgatagttt
gagttcttctactcaggcaagtgatgttattactaatcaaagaagtattgctacaacggttaatttgcgtgatggacagactctttta
ctcggtggcctcactgattataaaaacacttctcaagattctggcgtaccgttcctgtctaaaatccctttaatcggcctcctgttta
gctcccgctctgattccaacgaggaaagcacgttatacgtgctcgtcaaagcaaccatagtacgcgccctgtagcggcgcattaagcg
cggcgggtgtggtggttacgcgcagcgtgaccgctacacttgccagcgccctagcgcccgctcctttcgctttcttcccttcctttct
cgccacgttegccggctttccccgtcaagctctaaatcgggggctccctttagggttccgatttagtgctttacggcacctcgacccc
aaaaaacttgatttgggtgatggttcacgtagtgggccatcgccctgatagacggtttttcgccctttgacgttggagtccacgttct
ttaatagtggactcttgttccaaactggaacaacactcaaccctatctcgggctattcttttgatttataagggattttgccgatttc
ggtaaaaaatgagctgatttaacaaaaatttaatgcgaattttaacaaaatattaacgtttacaatttaaatatttgcttatacaatc
ttcctgtttttggggcttttctgattatcaaccggggtacatatgattgacatgctagttttacgattaccgttcatcgattctcttg
tttgctccagactctcaggcaatgacctgatagcctttgtagatctctcaaaaatagctaccctctccggcattaatttatcagctag
aacggttgaatatcatattgatggtgatttgactgtctccggcctttctcacccttttgaatctttacctacacattactcaggcatt
gcatttaaaatatatgagggttctaaaaatttttatccttgcgttgaaataaaggcttctcccgcaaaagtattacagggtcataatg
tttttggtacaaccgatttagctttatgctctgaggctttattgcttaattttgctaattctttgccttgcctgtatgatttattgga
cgtt
Artificial Amino Acid Sequence
SEQ ID NO: 4
FLVIR
M13 pIII Nucleic Acid Sequence for 1X104
SEQ ID NO: 5
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagggtggcggttctgagggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgctggcggcggctctggtggtggttctggtggcggctctgagggtggtggctctga
gggtggcggttctgagggtggcggctctgagggaggcggttccggtggtggctctggttccggtgattttgattatgaaaagatggca
aacgctaataagggggctatgaccgaaaatgccgatgaaaacgcgctacagtctgacgctaaaggcaaacttgattctgtcgctactg
attacggtgctgctatcgatggtttcattggtgacgtttccggccttgctaatggtaatggtgctactggtgattttgctggctctaa
ttcccaaatggctcaagtcggtgacggtgataattcacctttaatgaataatttccgtcaatatttaccttccctccctcaatcggtt
gaatgtcgcccttttgtctttagcgctggtaaaccatatgaattttctattgattgtgacaaaataaacttattccgtggtgtctttg
cgtttcttttatatgttgccacctttatgtatgtattttctacgtttgctaacatactgcgtaataaggagtct
M13 pIII Amino Acid Sequence for 1X104
SEQ ID NO: 6
AETVESCLAKSHTENSFTNVWKDDKTLDRYANYEGCLWNATGVVVCTGDETQCYGTWVPIGLAIPENEGGGSEGGGSEGGGSEGGGTK
PPEYGDTPIPGYTYINPLDGTYPPGTEQNPANPNPSLEESQPLNTFMFQNNRFRNRQGALTVYTGTVTQGTDPVKTYYQYTPVSSKAM
YDAYWNGKFRDCAFHSGFNEDLFVCEYQGQSSDLPQPPVNAGGGSGGGSGGGSEGGGSEGGGSEGGGSEGGGSGGGSGSGDFDYEKMA
NANKGAMTENADENALQSDAKGKLDSVATDYGAAIDGFIGDVSGLANGNGATGDFAGSNSQMAQVGDGDNSPLMNNFRQYLPSLPQSV
ECRPFVFSAGKPYEFSIDCDKINLFRGVFAFLLYVATFMYVFSTFANILRNKES
GS1 Linker Nucleic Acid Sequence for 1X104
SEQ ID NO: 7
ggtggtggctctgagggtggcggttctgagggtggcggttctgagggtggcggta
GS1 Linker Amino Acid Sequence for 1X104
SEQ ID NO: 8
GGGSEGGGSEGGGSEGGG
GS2 Linker Nucleic Acid Sequence for 1X104
SEQ ID NO: 9
ggcggcggctctggtggtggttctggtggcggctctgagggtggtggctctgagggtggcggttctgagggtggcggctctgagggag
gcggttccggtggtggctctggttccggt
GS2 linker Amino Acid Sequence for 1X104
SEQ ID NO: 10
GGGSGGGSGGGSEGGGSEGGGSEGGGSEGGGSGGGSGSG
pIII Nucleic Acid Sequence for M13 8 + 11 bacteriophage vector
SEQ ID NO: 11
gccgagacagtggagagctgcctggccaagtcgcacaccgagaacagcttcaccaatgtttggaaggatgataagaccctggaccgct
atgccaattacgaaggttgcttatggaacgcaaccggtgtggttgtgtgcacaggcgatgagacccaatgctatggcacctgggtgcc
gatcggtctggcaattccggagaacgaaggcggaggtagcgaaggaggtggaagtgaaggcggaggatcggaagggggtggcacaaag
ccaccagaatatggagacaccccgattccaggttacacctacattaatccgctggatggtacataccctccaggcaccgaacagaatc
cggcaaacccgaacccgagcctggaagaaagccaaccgctgaacacatttatgttccaaaacaaccgttttcgtaaccgtcaaggagc
cctgaccgtatacaccggtacagtgacccagggtacagatccggtgaagacctactatcaatatacaccggttagcagcaaggcaatg
tacgatgcatattggaatggcaagtttcgtgattgtgcatttcatagcggtttcaacgaagacccgtttgtgtgcgaataccagggtc
agagcagcgatttaccgcagccaccggttaacgcaggtggtggaagcggagggggaagtggcggtgggtcagaaggcggaggatcgga
aggaggtgggagtgaaggagggggaagcgaaggagggggatcaggaggtggtagcggaagtggcgacttcgactacgagaagatggcc
aatgcaaacaaaggcgcaatgacagagaacgcagacgagaatgcactgcaaagtgatgcaaagggtaagctggacagcgttgcaaccg
actatggagcagcaattgacggctttatcggagatgtcagcggtctggcgaacggcaacggagcaacaggcgacttcgcaggtagcaa
cagccagatggcacaggttggagatggcgacaacagtccgctgatgaacaactttcgccagtacctgccgagtctgccacaaagcgtc
gagtgccgtccgtacgttttcggtgcaggcaagccgtacgagttcagcatcgactgcgataagattaatctttttcgcggagttttcg
cattcctgctgtacgtggcaacgttcatgtacgttttcagcaccttcgccaatatcttacgcaacaaagaaagc
Artificial Amino Acid Sequence (NNJA_1)
SEQ ID NO: 12
MSTRGPTPA
Artificial Amino Acid Sequence (NNJA_2)
SEQ ID NO: 13
MTAPAPGLQ
Artificial Amino Acid Sequence (NNJA_3)
SEQ ID NO: 14
MTSSSDLRL
Artificial Amino Acid Sequence (NNJA_4)
SEQ ID NO: 15
LSSRTTYQG
Artificial Amino Acid Sequence (NNJA_5)
SEQ ID NO: 16
MTSKNTQIG
Artificial Amino Acid Sequence (NNJA_6)
SEQ ID NO: 17
MSHVGFETT
Artificial Amino Acid Sequence (NNJA_7)
SEQ ID NO: 18
MQPMGSTAS
Artificial Amino Acid Sequence (NNJA_8)
SEQ ID NO: 19
MTPSRLPPS
Artificial Amino Acid Sequence (NNJA_9)
SEQ ID NO: 20
MSKQNYHVV
Artificial Amino Acid Sequence (NNJA_10)
SEQ ID NO: 21
MAGYRSAVN
Artificial Amino Acid Sequence (NNJA_11)
SEQ ID NO: 22
MTTKHVATQ
Artificial Amino Acid Sequence (NNJA_12)
SEQ ID NO: 23
MTRTSTEPT
Artificial Amino Acid Sequence (NNJA_13)
SEQ ID NO: 24
MTTPNPKVR
Artificial Amino Acid Sequence (NNJA_14)
SEQ ID NO: 25
LTRQTNLEV
Artificial Amino Acid Sequence (NNJA_15)
SEQ ID NO: 26
SSRPPIVTP
Artificial Amino Acid Sequence (NNJA_16)
SEQ ID NO: 27
YTRPMSAPN
Artificial Amino Acid Sequence (NNJA_17)
SEQ ID NO: 28
FTSPPTEPR
Artificial Amino Acid Sequence (NNJA_18)
SEQ ID NO: 29
MGNWTPHGT
Artificial Amino Acid Sequence (NNJA_19)
SEQ ID NO: 30
MTSSRDAPA
Artificial Amino Acid Sequence (NNJA_20)
SEQ ID NO: 31
MSRQSVHTT
Artificial Amino Acid Sequence (NNJA_21)
SEQ ID NO: 32
FTSQTKVAM
Artificial Amino Acid Sequence (NNJA_22)
SEQ ID NO: 33
MSRPSSTLL
Artificial Amino Acid Sequence (NNJA_23)
SEQ ID NO: 34
MSTPLDRTN
Artificial Amino Acid Sequence (NNJA_24)
SEQ ID NO: 35
MQMATSTPA
Artificial Amino Acid Sequence (NNJA_25)
SEQ ID NO: 36
MSKPTRLPV
Artificial Amino Acid Sequence (NNJA_26)
SEQ ID NO: 37
LTTTRSLPS
Artificial Amino Acid Sequence (NNJA_27)
SEQ ID NO: 38
MGSPPTYRP
Artificial Amino Acid Sequence (NNJA_28)
SEQ ID NO: 39
MSLKSTPHP
Artificial Amino Acid Sequence (NNJA_29)
SEQ ID NO: 40
MSTAPPSRT
Artificial Amino Acid Sequence (NNJA_30)
SEQ ID NO: 41
MTSPNIAEP
Artificial Amino Acid Sequence (NNJA_31)
SEQ ID NO: 42
ASKVPPSGP
Artificial Amino Acid Sequence (NNJA_32)
SEQ ID NO: 43
AASTRPPQL
Artificial Amino Acid Sequence (NNJA_33)
SEQ ID NO: 44
MSQRLSHHD
Artificial Amino Acid Sequence (NNJA_34)
SEQ ID NO: 45
RLAKAPPVS
Artificial Amino Acid Sequence (NNJA_35)
SEQ ID NO: 46
MSRTNTTVN
Artificial Amino Acid Sequence (NNJA_36)
SEQ ID NO: 47
MSNPLSLPA
Artificial Amino Acid Sequence (NNJA_37)
SEQ ID NO: 48
MSNTFHRSE
Nucleic acid sequence of Engineered pIII including an amino
acid sequence (Clone_H4) for 1X104
SEQ ID NO: 49
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagggtggcggttctgagggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgctggcggcggctcttttttagttattagaggtggtggttctggtggcggctctga
gggtggtggctctgagtttttagttattagaggtggcggttctgagggtggcggctctgagggaggcggttccggtggtggctctggt
tccggtgattttgattatgaaaagatggcaaacgctaataagggggctatgaccgaaaatgccgatgaaaacgcgctacagtctgacg
ctaaaggcaaacttgattctgtcgctactgattacggtgctgctatcgatggtttcattggtgacgtttccggccttgctaatggtaa
tggtgctactggtgattttgctggctctaattcccaaatggctcaagtcggtgacggtgataattcacctttaatgaataatttccgt
caatatttaccttccctccctcaatcggttgaatgtcgcccttttgtctttagcgctggtaaaccatatgaattttctattgattgtg
acaaaataaacttattccgtggtgtctttgcgtttcttttatatgttgccacctttatgtatgtattttctacgtttgctaacatact
gcgtaataaggagtct
Nucleic acid sequence of Engineered pIII including an amino
acid sequence (Clone_G3) for 1X104
SEQ ID NO: 50
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagtttttagttattagaggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgctggcggcggctctggtggtggttctggtggcggctctgagggtggtggctctga
gtttttagttattagaggtggcggttctgagggtggcggctctgagggaggcggttccggtggtggctctggttccggtgattttgat
tatgaaaagatggcaaacgctaataagggggctatgaccgaaaatgccgatgaaaacgcgctacagtctgacgctaaaggcaaacttg
attctgtcgctactgattacggtgctgctatcgatggtttcattggtgacgtttccggccttgctaatggtaatggtgctactggtga
ttttgctggctctaattcccaaatggctcaagtcggtgacggtgataattcacctttaatgaataatttccgtcaatatttaccttcc
ctccctcaatcggttgaatgtcgcccttttgtctttagcgctggtaaaccatatgaattttctattgattgtgacaaaataaacttat
tccgtggtgtctttgcgtttcttttatatgttgccacctttatgtatgtattttctacgtttgctaacatactgcgtaataaggagtc
t
Nucleic acid sequence of Engineered pIII including an amino
acid sequence (Clone_G1) for 1X104
SEQ ID NO: 51
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagtttttagttattagaggtggcggttctgagggtggcggttctgag
ggtggcggtactaaacctcctgagtacggtgatacacctattccgggctatacttatatcaaccctctcgacggcacttatccgcctg
gtactgagcaaaaccccgctaatcctaatccttctcttgaggagtctcagcctcttaatactttcatgtttcagaataataggttccg
aaataggcagggggcattaactgtttatacgggcactgttactcaaggcactgaccccgttaaaacttattaccagtacactcctgta
tcatcaaaagccatgtatgacgcttactggaacggtaaattcagagactgcgctttccattctggctttaatgaggatttatttgttt
gtgaatatcaaggccaatcgtctgacctgcctcaacctcctgtcaatgctggcggcggctctggtggtggttctggtggcggctctga
gggtggtggctctgagggtggcggttctgagtttttagttattagaggtggcggctctgagggaggcggttccggtggtggctctggt
tccggtgattttgattatgaaaagatggcaaacgctaataagggggctatgaccgaaaatgccgatgaaaacgcgctacagtctgacg
ctaaaggcaaacttgattctgtcgctactgattacggtgctgctatcgatggtttcattggtgacgtttccggccttgctaatggtaa
tggtgctactggtgattttgctggctctaattcccaaatggctcaagtcggtgacggtgataattcacctttaatgaataatttccgt
caatatttaccttccctccctcaatcggttgaatgtcgcccttttgtctttagcgctggtaaaccatatgaattttctattgattgtg
acaaaataaacttattccgtggtgtctttgcgtttcttttatatgttgccacctttatgtatgtattttctacgtttgctaacatact
gcgtaataaggagtct
Nucleic acid sequence of Engineered pIII including an amino
acid sequence (Clone_D4) for 1X104
SEQ ID NO: 52
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagggtggcggttctgagggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgctggcggcggctcttttttagttattagaggtggtggttctggtggcggctctga
gggtggtggctctgagggtggcggttctgagggtggcggctctgagggaggcggttcctttttagttattagaggtggtggctctggt
tccggtgattttgattatgaaaagatggcaaacgctaataagggggctatgaccgaaaatgccgatgaaaacgcgctacagtctgacg
ctaaaggcaaacttgattctgtcgctactgattacggtgctgctatcgatggtttcattggtgacgtttccggccttgctaatggtaa
tggtgctactggtgattttgctggctctaattcccaaatggctcaagtcggtgacggtgataattcacctttaatgaataatttccgt
caatatttaccttccctccctcaatcggttgaatgtcgcccttttgtctttagcgctggtaaaccatatgaattttctattgattgtg
acaaaataaacttattccgtggtgtctttgcgtttcttttatatgttgccacctttatgtatgtattttctacgtttgctaacatact
gcgtaataaggagtct
Nucleic acid sequence of Engineered pIII including an amino
acid sequence (Clone_F4) for 1X104
SEQ ID NO: 53
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagtttttagttattagaggtggcggttctgagggtggcggttctgag
ggtggcggtactaaacctcctgagtacggtgatacacctattccgggctatacttatatcaaccctctcgacggcacttatccgcctg
gtactgagcaaaaccccgctaatcctaatccttctcttgaggagtctcagcctcttaatactttcatgtttcagaataataggttccg
aaataggcagggggcattaactgtttatacgggcactgttactcaaggcactgaccccgttaaaacttattaccagtacactcctgta
tcatcaaaagccatgtatgacgcttactggaacggtaaattcagagactgcgctttccattctggctttaatgaggatttatttgttt
gtgaatatcaaggccaatcgtctgacctgcctcaacctcctgtcaatgctggcggcggctctggtggtggttctggtggcggctctga
gtttttagttattagaggtggtggctctgagggtggcggttctgagggtggcggctctgagggaggcggttccggtggtggctctggt
tccggtgattttgattatgaaaagatggcaaacgctaataagggggctatgaccgaaaatgccgatgaaaacgcgctacagtctgacg
ctaaaggcaaacttgattctgtcgctactgattacggtgctgctatcgatggtttcattggtgacgtttccggccttgctaatggtaa
tggtgctactggtgattttgctggctctaattcccaaatggctcaagtcggtgacggtgataattcacctttaatgaataatttccgt
caatatttaccttccctccctcaatcggttgaatgtcgcccttttgtctttagcgctggtaaaccatatgaattttctattgattgtg
acaaaataaacttattccgtggtgtctttgcgtttcttttatatgttgccacctttatgtatgtattttctacgtttgctaacatact
gcgtaataaggagtct
Nucleic acid sequence of Engineered pIII including an amino
acid sequence (Clone_B4) for 1X104
SEQ ID NO: 54
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagtttttagttattagaggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgcttttttagttattagaggcggcggctctggtggtggttctggtggcggctctga
gggtggtggctctgagggtggcggttctgagtttttagttattagaggtggcggctctgagggaggcggttccggtggtggctctggt
tccggtgattttgattatgaaaagatggcaaacgctaataagggggctatgaccgaaaatgccgatgaaaacgcgctacagtctgacg
ctaaaggcaaacttgattctgtcgctactgattacggtgctgctatcgatggtttcattggtgacgtttccggccttgctaatggtaa
tggtgctactggtgattttgctggctctaattcccaaatggctcaagtcggtgacggtgataattcacctttaatgaataatttccgt
caatatttaccttccctccctcaatcggttgaatgtcgcccttttgtctttagcgctggtaaaccatatgaattttctattgattgtg
acaaaataaacttattccgtggtgtctttgcgtttcttttatatgttgccacctttatgtatgtattttctacgtttgctaacatact
gcgtaataaggagtct
Nucleic acid sequence of Engineered pIII including an amino
acid sequence (Clone_A4) for 1X104
SEQ ID NO: 55
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagtttttagttattagaggtggcggttctgagggtggcggttctgag
ggtggcggtactaaacctcctgagtacggtgatacacctattccgggctatacttatatcaaccctctcgacggcacttatccgcctg
gtactgagcaaaaccccgctaatcctaatccttctcttgaggagtctcagcctcttaatactttcatgtttcagaataataggttccg
aaataggcagggggcattaactgtttatacgggcactgttactcaaggcactgaccccgttaaaacttattaccagtacactcctgta
tcatcaaaagccatgtatgacgcttactggaacggtaaattcagagactgcgctttccattctggctttaatgaggatttatttgttt
gtgaatatcaaggccaatcgtctgacctgcctcaacctcctgtcaatgctggcggcggctctggtggtggttctggtggcggctctga
gggtggtggctctgagggtggcggttctgagggtggcggctctgagggaggcggttccggtggtggctcttttttagttattagaggt
tccggtgattttgattatgaaaagatggcaaacgctaataagggggctatgaccgaaaatgccgatgaaaacgcgctacagtctgacg
ctaaaggcaaacttgattctgtcgctactgattacggtgctgctatcgatggtttcattggtgacgtttccggccttgctaatggtaa
tggtgctactggtgattttgctggctctaattcccaaatggctcaagtcggtgacggtgataattcacctttaatgaataatttccgt
caatatttaccttccctccctcaatcggttgaatgtcgcccttttgtctttagcgctggtaaaccatatgaattttctattgattgtg
acaaaataaacttattccgtggtgtctttgcgtttcttttatatgttgccacctttatgtatgtattttctacgtttgctaacatact
gcgtaataaggagtct
Nucleic acid sequence of Engineered pIII including an amino
acid sequence (Clone_F1) for 1X104
SEQ ID NO: 56
gctgaaactgttgaaagttgtttagcaaaatcccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagtttttagttattagaggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgctggcggcggctctggtggtggttcttttttagttattagaggtggcggctctga
gggtggtggctctgagggtggcggttctgagggtggcggctctgagggaggcggttccggtggtggctctggttccggtgattttgat
tatgaaaagatggcaaacgctaataagggggctatgaccgaaaatgccgatgaaaacgcgctacagtctgacgctaaaggcaaacttg
attctgtcgctactgattacggtgctgctatcgatggtttcattggtgacgtttccggccttgctaatggtaatggtgctactggtga
ttttgctggctctaattcccaaatggctcaagtcggtgacggtgataattcacctttaatgaataatttccgtcaatatttaccttcc
ctccctcaatcggttgaatgtcgcccttttgtctttagcgctggtaaaccatatgaattttctattgattgtgacaaaataaacttat
tccgtggtgtctttgcgtttcttttatatgttgccacctttatgtatgtattttctacgtttgctaacatactgcgtaataaggagtc
t
Nucleic Acid Sequence for 8 + 11 vector
SEQ ID NO: 57
aatgctactactattagtagaattgatgccaccttttcagctcgcgccccaaatgaaaatatagctaaacaggttattgaccatttgc
gaaatgtatctaatggtcaaactaaatctactcgttcgcagaattgggaatcaactgttatatggaatgaaacttccagacaccgtac
tttagttgcatatttaaaacatgttgagctacagcattatattcagcaattaagctctaagccatctgcaaaaatgacctcttatcaa
aaggagcaattaaaggtactctctaatcctgacctgttggagtttgcttccggtctggttcgctttgaagctcgaattaaaacgcgat
atttgaagtctttcgggcttcctcttaatctttttgatgcaatccgctttgcttctgactataatagtcagggtaaagacctgatttt
tgatttatggtcattctcgttttctgaactgtttaaagcatttgagggggattcaatgaatatttatgacgattccgcagtattggac
gctatccagtctaaacattttactgttaccccctctggcaaaacttcttttgcaaaagcctctcgctattttggtttttatcgtcgtc
tggtaaacgagggttatgatagtgttgctcttactatgcctcgtaattccttttggcgttatgtatctgcattagttgaatgtggtat
tcctaaatctcaactgatgaatctttctacctgtaataatgttgttccgttagttcgttttattaacgtagatttttcttcccaacgt
cctgactggtataatgagccagttcttaaaatcgcataaggtaattcacaatgattaaagttgaaattaaaccatctcaagcccaatt
tactactcgttctggtgtttctcgtcagggcaagccttattcactgaatgagcagctttgttacgttgatttgggtaatgaatatccg
gttcttgtcaagattactcttgatgaaggtcagccagcctatgcgcctggtctgtacaccgttcatctgtcctctttcaaagttggtc
agttcggttcccttatgattgaccgtctgcgcctcgttccggctaagtaacatggagcaggtcgcggatttcgacacaatttatcagg
cgatgatacaaatctccgttgtactttgtttcgcgcttggtataatcgctgggggtcaaagatgagtgttttagtgtattcttttgcc
tctttcgttttaggttggtgccttcgtagtggcattacgtattttacccgtttaatggaaacttcctcatgaaaaagtctttagtcct
caaagcctctgtagccgttgctaccctcgttccgatgctgtctttcgctgctgagggtgacgatcccgcaaaagcggcctttaactcc
ctgcaagcctcagcgaccgaatatatcggttatgcgtgggcgatggttgttgtcattgtcggcgcaactatcggtatcaagctgttta
agaaattcacctcgaaagcaagctgataaaccgatacaattaaaggctccttttggagccttttttttggagattttcaacgtgaaaa
aattattattcgcaattcctttagttgttcctttctattctcactccgctgaaactgttgaaagttgtccggcaaaaccccatacaga
aaattcatttactaacgtctggaaagacgacaaaactttagatcgttacgctaactatgagggctgtctgtggaatgctacaggcgtt
gtagtttgtactggtgacgaaactcagtgttacggtacatgggttcctattgggcttgctatccctgaaaatgagggtggtggctctg
aggagggtggcggttctgagggtggcggtactaaacctcctgagtacggtgatacacctattccgggctatacttatatcaaccctct
cgacggcacttatccgcctggtactgagcaaaaccccgctaatcctaatccttctcttgaggagtctcagcctcttaatactttcatg
tttcagaataataggttccgaaataggcagggggcattaactgtttatacgggcactgttactcaaggcactgaccccgttaaaactt
attaccagtacactcctgtatcatcaaaagccatgtatgacgcttactggaacggtaaattcagagactgcgctttccattctggctt
taatgaggatttatttgtttgtgaatatcaaggccaatcgtctgacctgcctcaacctcctgtcaatgctggcggcggctctggtggt
ggttctggtggcggttctgagggtggtggctctgagggtggcggttctgagggtggcggctctgagggaggcggttccggtggtggct
ctggttccggtgattttgattatgaaaagatggcaaacgctaataagggggctatgaccgaaaatgccgatgaaaacgcgctacagtc
tgacgctaaaggcaaacttgattctgtcgctactgattacggtgctgctatcgatggtttcattggtgacgtttccggccttgctaat
ggtaatggtgctactggtgattttgctggctctaattcccaaatggctcaagtcggtgacggtgataattcacctttaatgaataatt
tccgtcaatatttaccttccctccctcaatcggttgaatgtcgcccttttgtctttggcgctggtaaaccatatgaattttctattga
ttgtgacaaaataaacttattccgtggtgtctttgcgtttcttttatatgttgccacctttatgtatgtattttcgacgtttgctaac
atactgcgtaataaggagtcttaatcatgccagttcttttgggtattccgttattattgcgtttcctcggtttccttctggtaacttt
gttcggctatctgcttacttttcttaaaaagggcttcggtaagatagctattgctatttcattgtttcttgctcttattattgggctt
aactcaattcttgtgggttatctctctgatattagcgctcaattaccctctgactttgttcagggtgttcagttaattctcccgtcta
atgcgcttccctgtttttatgttattctctctgtaaaggctgctattttcatttttgacgttaaacaaaaaatcgtttcttatttgga
ttgggataaataatatggctgtttattttgtaactggcaaattaggctctggaaagacgctcgttagcgttggtaagattcaggataa
aattgtagctgggtgcaaaatagcaactaatcttgatttaaggcttcaaaacctcccgcaagtcgggaggttcgctaaaacgcctcgc
gttcttagaataccggataagccttctatatctgatttgcttgctattgggcgcggtaatgattcctacgatgaaaataaaaacggct
tgcttgttctcgatgagtgcggtacttggtttaatacccgttcttggaatgataaggaaagacagccgattattgattggtttctaca
tgctcgtaaattaggatgggatattatttttcttgttcaggacttatctattgttgataaacaggcgcgttctgcattagctgaacat
gttgtttattgtcgtcgtctggacagaattactttaccttttgtcggtactttatattctcttattactggctcgaaaatgcctctgc
ctaaattacatgttggcgttgttaaatatggcgattctcaattaagccctactgttgagcgttggctttatactggtaagaatttgta
taacgcatatgatactaaacaggctttttctagtaattatgattccggtgtttattcttatttaacgccttatttatcacacggtcgg
tatttcaaaccattaaatttaggtcagaagatgaagcttactaaaatatatttgaaaaagttttcacgcgttctttgtcttgcgattg
gatttgcatcagcatttacatatagttatataacccaacctaagccggaggttaaaaaggtagtctctcagacctatgattttgataa
attcactattgactcttctcagcgtcttaatctaagctatcgctatgttttcaaggattctaagggaaaattaattaatagcgacgat
ttacagaagcaaggttattcactcacatatattgatttatgtactgtttccattaaaaaaggtaattcaaatgaaattgttaaatgta
attaattttgttttcttgatgtttgtttcatcatcttcttttgctcaggtaattgaaatgaataattcgcctctgcgcgattttgtaa
cttggtattcaaagcaatcaggcgaatccgttattgtttctcccgatgtaaaaggtactgttactgtatattcatctgacgttaaacc
tgaaaatctacgcaatttctttatttctgttttacgtgcaaatgattttgatatggtaggttctaacccttccattattcagaagtat
aatccaaacaatcaggattatattgatgaattgccatcatctgataatcaggaatatgatgataattccgctccttctggtggtttct
ttgttccgcaaaatgataatgttactcaaacttttaaaattaataacgttcgggcaaaggatttaatacgagttgtcgaattgtttgt
aaagtctaatacttctaaatcctcaaatgtattatctattgacggctctaatctattagttgttagtgctcctaaagatattttagat
aaccttcctcaattcctttcaactgttgatttgccaactgaccagatattgattgagggtttgatatttgaggttcagcaaggtgatg
ctttagatttttcatttgctgctggctctcagcgtggcactgttgcaggcggtgttaatactgaccgcctcacctctgttttatcttc
tgctggtggttcgttcggtatttttaatggcgatgttttagggctatcagttcgcgcattaaagactaatagccattcaaaaatattg
tctgtgccacgtattcttacgctttcaggtcagaagggttctatctctgttggccagaatgtcccttttattactggtcgtgtgactg
gtgaatctgccaatgtaaataatccatttcagacgattgagcgtcaaaatgtaggtatttccatgagcgtttttcctgttgcaatggc
tggcggtaatattgttctggatattaccagcaaggccgatagtttgagttcttctactcaggcaagtgatgttattactaatcaaaga
agtattgctacaacggttaatttgcgtgatggacagactcttttactcggtggcctcactgattataaaaacacttctcaggattctg
gcgtaccgttcctgtctaaaatccctttaatcggcctcctgtttagctcccgctctgattctaacgaggaaagcacgttatacgtgct
cgtcaaagcaaccatagtacgcgccctgtagcggcgcattaagcgcggcgggtgtggtggttacgcgcagcgtgaccgctacacttgc
cagcgccctagcgcccgctcctttcgctttcttcccttcctttctcgccacgttcgccggctttccccgtcaagctctaaatcggggg
ctccctttagggttccgatttagtgctttacggcacctcgaccccaaaaaacttgatttgggtgatggttcacgtagtgggccatcgc
cctgatagacggtttttcgccctttgacgttggagtccacgttctttaatagtggactcttgttccaaactggaacaacactcaaccc
tatctcgggctattcttttgatttataagggattttgccgatttcggaaccaccatcacacaggattttcgcctgctggggcaaacca
gcgtggaccgcttgctgcaactctctcagggccaggcggtgaagggcaatcagctgttgcccgtctcgctggtgaaaagaaaaaccac
cctggcgcccaatacgcaaaccgcctctccccgcgcgttggccgattcattaatgcagctggcacgacaggtttcccgactggaaagc
gggcagtgagcgcaacgcaattaatgtgagttagctcactcattaggcaccccaggcttgacactttatgcttccggctcgtataatg
tgtggaattgtgagcggataacaatttcacacgccaaggagacagtcataatgaaatacctattgcctacggcagccgctggattgtt
attactcgctgcccaaccagccatggcctaacggggggaattcggggggccctttaaagaattcgcatacgaattctttaaagggccc
cccgaattccccccgttataacggcggaggatctggcgagcaaaagctcattagtgaagaggatcttgagacagtggagagctgcctg
gccaagccgcacaccgagaacagcttcaccaatgtttggaaggatgataagaccctggaccgctatgccaattacgaaggttgcttat
ggaacgcaaccggtgtggttgtgtgcacaggcgatgagacccaatgctatggcacctgggtgccgatcggtctggcaattccggagaa
cgaaggcggaggtagcgaaggaggtggaagtgaaggcggaggatcggaagggggtggcacaaagccaccagaatatggagacaccccg
attccaggttacacctacattaatccgctggatggtacataccctccaggcaccgaacagaatccggcaaacccgaacccgagcctgg
aagaaagccaaccgctgaacacatttatgttccaaaacaaccgttttcgtaaccgtcaaggagccctgaccgtatacaccggtacagt
gacccagggtacagatccggtgaagacctactatcaatatacaccggttagcagcaaggcaatgtacgatgcatattggaatggcaag
tttcgtgattgtgcatttcatagcggtttcaacgaagacccgtttgtgtgcgaataccagggtcagagcagcgatttaccgcagccac
cggttaacgcaggtggtggaagcggagggggaagtggcggtgggtcagaaggcggaggatcggaaggaggtgggagtgaaggaggggg
aagcgaaggagggggatcaggaggtggtagcggaagtggcgacttcgactacgagaagatggccaatgcaaacaaaggcgcaatgaca
gagaacgcagacgagaatgcactgcaaagtgatgcaaagggtaagctggacagcgttgcaaccgactatggagcagcaattgacggct
ttatcggagatgtcagcggtctggcgaacggcaacggagcaacaggcgacttcgcaggtagcaacagccagatggcacaggttggaga
tggcgacaacagtccgctgatgaacaactttcgccagtacctgccgagtctgccacaaagcgtcgagtgccgtccgtacgttttcggt
gcaggcaagccgtacgagttcagcatcgactgcgataagattaatctttttcgcggagttttcgcattcctgctgtacgtggcaacgt
tcatgtacgttttcagcaccttcgccaatatcttacgcaacaaagaaagctaagcaatagcgaagaggcccgcaccgatcgcccttcc
caacagttgcgcagcctgaatggcgaatggcgctttgcctggtttccggcaccagaagcggtgccggaaagctggctggagtgcgatc
ttcctgaggccgatactgtcgtcgtcccctcaaactggcagatgcacggttacgatgcgcccatctacaccaacgtgacctatcccat
tacggtcaatccgccgtttgttcccacggagaatccgacgggttgttactcgctcacatttaatgttgatgaaagctggctacaggaa
ggccagacgcgaattatttttgatggcgttcctattggttaaaaaatgagctgatttaacaaaaatttaatgcgaattttaacaaaat
attaacgtttacaatttaaatatttgcttatacaatcttcctgtttttggggcttttctgattatcaaccggggtacatatgattgac
atgctagttttacgattaccgttcatcgattctcttgtttgctccagactctcaggcaatgacctgatagcctttgtagatctctcaa
aaatagctaccctctccggcattaatttatcagctagaacggttgaatatcatattgatggtgatttgactgtctccggcctttctca
cccttttgaatctttacctacacattactcaggcattgcatttaaaatatatgagggttctaaaaatttttatccttgcgttgaaata
aaggcttctcccgcaaaagtattacagggtcataatgtttttggtacaaccgatttagctttatgctctgaggctttattgcttaatt
ttgctaattctttgccttgcctgtatgatttattggacgtt
H4 bacteriophage Nucleic Acid Sequence for 8P + 11P vector
SEQ ID NO: 58
aatgctactactattagtagaattgatgccaccttttcagctcgcgccccaaatgaaaatatagctaaacaggttattgaccatttgc
gaaatgtatctaatggtcaaactaaatctactcgttcgcagaattgggaatcaactgttatatggaatgaaacttccagacaccgtac
tttagttgcatatttaaaacatgttgagctacagcattatattcagcaattaagctctaagccatctgcaaaaatgacctcttatcaa
aaggagcaattaaaggtactctctaatcctgacctgttggagtttgcttccggtctggttcgctttgaagctcgaattaaaacgcgat
atttgaagtctttcgggcttcctcttaatctttttgatgcaatccgctttgcttctgactataatagtcagggtaaagacctgatttt
tgatttatggtcattctcgttttctgaactgtttaaagcatttgagggggattcaatgaatatttatgacgattccgcagtattggac
gctatccagtctaaacattttactgttaccccctctggcaaaacttcttttgcaaaagcctctcgctattttggtttttatcgtcgtc
tggtaaacgagggttatgatagtgttgctcttactatgcctcgtaattccttttggcgttatgtatctgcattagttgaatgtggtat
tcctaaatctcaactgatgaatctttctacctgtaataatgttgttccgttagttcgttttattaacgtagatttttcttcccaacgt
cctgactggtataatgagccagttcttaaaatcgcataaggtaattcacaatgattaaagttgaaattaaaccatctcaagcccaatt
tactactcgttctggtgtttctcgtcagggcaagccttattcactgaatgagcagctttgttacgttgatttgggtaatgaatatccg
gttcttgtcaagattactcttgatgaaggtcagccagcctatgcgcctggtctgtacaccgttcatctgtcctctttcaaagttggtc
agttcggttcccttatgattgaccgtctgcgcctcgttccggctaagtaacatggagcaggtcgcggatttcgacacaatttatcagg
cgatgatacaaatctccgttgtactttgtttcgcgcttggtataatcgctgggggtcaaagatgagtgttttagtgtattcttttgcc
tctttcgttttaggttggtgccttcgtagtggcattacgtattttacccgtttaatggaaacttcctcatgaaaaagtctttagtcct
caaagcctctgtagccgttgctaccctcgttccgatgctgtctttcgctgctgagggtgacgatcccgcaaaagcggcctttaactcc
ctgcaagcctcagcgaccgaatatatcggttatgcgtgggcgatggttgttgtcattgtcggcgcaactatcggtatcaagctgttta
agaaattcacctcgaaagcaagctgataaaccgatacaattaaaggctccttttggagccttttttttggagattttcaacgtgaaaa
aattattattcgcaattcctttagttgttcctttctattctcactccgctgaaactgttgaaagttgtccggcaaaaccccatacaga
aaattcatttactaacgtctggaaagacgacaaaactttagatcgttacgctaactatgagggctgtctgtggaatgctacaggcgtt
gtagtttgtactggtgacgaaactcagtgttacggtacatgggttcctattgggcttgctatccctgaaaatgagggtggtggctctg
aggagggtggcggttctgagggtggcggtactaaacctcctgagtacggtgatacacctattccgggctatacttatatcaaccctct
cgacggcacttatccgcctggtactgagcaaaaccccgctaatcctaatccttctcttgaggagtctcagcctcttaatactttcatg
tttcagaataataggttccgaaataggcagggggcattaactgtttatacgggcactgttactcaaggcactgaccccgttaaaactt
attaccagtacactcctgtatcatcaaaagccatgtatgacgcttactggaacggtaaattcagagactgcgctttccattctggctt
taatgaggatttatttgtttgtgaatatcaaggccaatcgtctgacctgcctcaacctcctgtcaatgctggcggcggctctttttta
gttattagaggtggtggttctggtggcggctctgagggtggtggctctgagtttttagttattagaggtggcggttctgagggtggcg
gctctgagggaggcggttccggtggtggctctggttccggtgattttgattatgaaaagatggcaaacgctaataagggggctatgac
cgaaaatgccgatgaaaacgcgctacagtctgacgctaaaggcaaacttgattctgtcgctactgattacggtgctgctatcgatggt
ttcattggtgacgtttccggccttgctaatggtaatggtgctactggtgattttgctggctctaattcccaaatggctcaagtcggtg
acggtgataattcacctttaatgaataatttccgtcaatatttaccttccctccctcaatcggttgaatgtcgcccttttgtctttgg
cgctggtaaaccatatgaattttctattgattgtgacaaaataaacttattccgtggtgtctttgcgtttcttttatatgttgccacc
tttatgtatgtattttcgacgtttgctaacatactgcgtaataaggagtcttaatcatgccagttcttttgggtattccgttattatt
gcgtttcctcggtttccttctggtaactttgttcggctatctgcttacttttcttaaaaagggcttcggtaagatagctattgctatt
tcattgtttcttgctcttattattgggcttaactcaattcttgtgggttatctctctgatattagcgctcaattaccctctgactttg
ttcagggtgttcagttaattctcccgtctaatgcgcttccctgtttttatgttattctctctgtaaaggctgctattttcatttttga
cgttaaacaaaaaatcgtttcttatttggattgggataaataatatggctgtttattttgtaactggcaaattaggctctggaaagac
gctcgttagcgttggtaagattcaggataaaattgtagctgggtgcaaaatagcaactaatcttgatttaaggcttcaaaacctcccg
caagtegggaggttcgctaaaacgcctegcgttcttagaataccggataagccttctatatctgatttgcttgctattgggcgcggta
atgattcctacgatgaaaataaaaacggcttgcttgttctcgatgagtgcggtacttggtttaatacccgttcttggaatgataagga
aagacagccgattattgattggtttctacatgctcgtaaattaggatgggatattatttttcttgttcaggacttatctattgttgat
aaacaggcgcgttctgcattagctgaacatgttgtttattgtcgtcgtctggacagaattactttaccttttgtcggtactttatatt
ctcttattactggctcgaaaatgcctctgcctaaattacatgttggcgttgttaaatatggcgattctcaattaagccctactgttga
gcgttggctttatactggtaagaatttgtataacgcatatgatactaaacaggctttttctagtaattatgattccggtgtttattct
tatttaacgccttatttatcacacggtcggtatttcaaaccattaaatttaggtcagaagatgaagcttactaaaatatatttgaaaa
agttttcacgcgttctttgtcttgcgattggatttgcatcagcatttacatatagttatataacccaacctaagccggaggttaaaaa
ggtagtctctcagacctatgattttgataaattcactattgactcttctcagcgtcttaatctaagctatcgctatgttttcaaggat
tctaagggaaaattaattaatagcgacgatttacagaagcaaggttattcactcacatatattgatttatgtactgtttccattaaaa
aaggtaattcaaatgaaattgttaaatgtaattaattttgttttcttgatgtttgtttcatcatcttcttttgctcaggtaattgaaa
tgaataattcgcctctgcgcgattttgtaacttggtattcaaagcaatcaggcgaatccgttattgtttctcccgatgtaaaaggtac
tgttactgtatattcatctgacgttaaacctgaaaatctacgcaatttctttatttctgttttacgtgcaaatgattttgatatggta
ggttctaacccttccattattcagaagtataatccaaacaatcaggattatattgatgaattgccatcatctgataatcaggaatatg
atgataattccgctccttctggtggtttctttgttccgcaaaatgataatgttactcaaacttttaaaattaataacgttcgggcaaa
ggatttaatacgagttgtcgaattgtttgtaaagtctaatacttctaaatcctcaaatgtattatctattgacggctctaatctatta
gttgttagtgctcctaaagatattttagataaccttcctcaattcctttcaactgttgatttgccaactgaccagatattgattgagg
gtttgatatttgaggttcagcaaggtgatgctttagatttttcatttgctgctggctctcagcgtggcactgttgcaggcggtgttaa
tactgaccgcctcacctctgttttatcttctgctggtggttcgttcggtatttttaatggcgatgttttagggctatcagttcgcgca
ttaaagactaatagccattcaaaaatattgtctgtgccacgtattcttacgctttcaggtcagaagggttctatctctgttggccaga
atgtcccttttattactggtcgtgtgactggtgaatctgccaatgtaaataatccatttcagacgattgagcgtcaaaatgtaggtat
ttccatgagcgtttttcctgttgcaatggctggcggtaatattgttctggatattaccagcaaggccgatagtttgagttcttctact
caggcaagtgatgttattactaatcaaagaagtattgctacaacggttaatttgcgtgatggacagactcttttactcggtggcctca
ctgattataaaaacacttctcaggattctggcgtaccgttcctgtctaaaatccctttaatcggcctcctgtttagctcccgctctga
ttctaacgaggaaagcacgttatacgtgctcgtcaaagcaaccatagtacgcgccctgtagcggcgcattaagcgcggcgggtgtggt
ggttacgcgcagcgtgaccgctacacttgccagcgccctagcgcccgctcctttcgctttcttcccttcctttctcgccacgttcgcc
ggctttccccgtcaagctctaaatcgggggctccctttagggttccgatttagtgctttacggcacctcgaccccaaaaaacttgatt
tgggtgatggttcacgtagtgggccatcgccctgatagacggtttttcgccctttgacgttggagtccacgttctttaatagtggact
cttgttccaaactggaacaacactcaaccctatctcgggctattcttttgatttataagggattttgccgatttcggaaccaccatca
cacaggattttcgcctgctggggcaaaccagcgtggaccgcttgctgcaactctctcagggccaggcggtgaagggcaatcagctgtt
gcccgtctcgctggtgaaaagaaaaaccaccctggcgcccaatacgcaaaccgcctctccccgcgcgttggccgattcattaatgcag
ctggcacgacaggtttcccgactggaaagcgggcagtgagcgcaacgcaattaatgtgagttagctcactcattaggcaccccaggct
tgacactttatgcttccggctcgtataatgtgtggaattgtgagcggataacaatttcacacgccaaggagacagtcataatgaaata
cctattgcctacggcagccgctggattgttattactcgctgcccaaccagccatggcctaacggggggaattcggggggccctttaaa
gaattcgcatacgaattctttaaagggccccccgaattccccccgttataacggcggaggatctggcgagcaaaagctcattagtgaa
gaggatcttgagacagtggagagctgcctggccaagccgcacaccgagaacagcttcaccaatgtttggaaggatgataagaccctgg
accgctatgccaattacgaaggttgcttatggaacgcaaccggtgtggttgtgtgcacaggcgatgagacccaatgctatggcacctg
ggtgccgatcggtctggcaattccggagaacgaaggcggaggtagcgaaggaggtggaagtgaaggcggaggatcggaagggggtggc
acaaagccaccagaatatggagacaccccgattccaggttacacctacattaatccgctggatggtacataccctccaggcaccgaac
agaatccggcaaacccgaacccgagcctggaagaaagccaaccgctgaacacatttatgttccaaaacaaccgttttcgtaaccgtca
aggagccctgaccgtatacaccggtacagtgacccagggtacagatccggtgaagacctactatcaatatacaccggttagcagcaag
gcaatgtacgatgcatattggaatggcaagtttcgtgattgtgcatttcatagcggtttcaacgaagacccgtttgtgtgcgaatacc
agggtcagagcagcgatttaccgcagccaccggttaacgcaggtggtggaagctttttagttattagaggagggggaagtggcggtgg
gtcagaaggcggaggatcggaatttttagttattagaggaggtgggagtgaaggagggggaagcgaaggagggggatcaggaggtggt
agcggaagtggcgacttcgactacgagaagatggccaatgcaaacaaaggcgcaatgacagagaacgcagacgagaatgcactgcaaa
gtgatgcaaagggtaagctggacagcgttgcaaccgactatggagcagcaattgacggctttatcggagatgtcagcggtctggcgaa
cggcaacggagcaacaggcgacttcgcaggtagcaacagccagatggcacaggttggagatggcgacaacagtccgctgatgaacaac
tttcgccagtacctgccgagtctgccacaaagcgtcgagtgccgtccgtacgttttcggtgcaggcaagccgtacgagttcagcatcg
actgcgataagattaatctttttcgcggagttttcgcattcctgctgtacgtggcaacgttcatgtacgttttcagcaccttcgccaa
tatcttacgcaacaaagaaagctaagcaatagcgaagaggcccgcaccgatcgcccttcccaacagttgcgcagcctgaatggcgaat
ggcgctttgcctggtttccggcaccagaagcggtgccggaaagctggctggagtgcgatcttcctgaggccgatactgtcgtcgtccc
ctcaaactggcagatgcacggttacgatgcgcccatctacaccaacgtgacctatcccattacggtcaatccgccgtttgttcccacg
gagaatccgacgggttgttactcgctcacatttaatgttgatgaaagctggctacaggaaggccagacgcgaattatttttgatggcg
ttcctattggttaaaaaatgagctgatttaacaaaaatttaatgcgaattttaacaaaatattaacgtttacaatttaaatatttgct
tatacaatcttcctgtttttggggcttttctgattatcaaccggggtacatatgattgacatgctagttttacgattaccgttcatcg
attctcttgtttgctccagactctcaggcaatgacctgatagcctttgtagatctctcaaaaatagctaccctctccggcattaattt
atcagctagaacggttgaatatcatattgatggtgatttgactgtctccggcctttctcacccttttgaatctttacctacacattac
tcaggcattgcatttaaaatatatgagggttctaaaaatttttatccttgcgttgaaataaaggcttctcccgcaaaagtattacagg
gtcataatgtttttggtacaaccgatttagctttatgctctgaggctttattgcttaattttgctaattctttgccttgcctgtatga
tttattggacgtt
pIII Nucleic Acid Sequence for Wildtype M13 bacteriophage
SEQ ID NO: 59
gctgaaactgttgaaagttgtttagcaaaatcccatacagaaaattcatttactaacgtctggaaagacgacaaaactttagatcgtt
acgctaactatgagggttgtctgtggaatgctacaggcgttgtagtttgtactggtgacgaaactcagtgttacggtacatgggttcc
tattgggcttgctatccctgaaaatgagggtggtggctctgagggggcggttctgagggtggcggttctgagggtggcggtactaaac
ctcctgagtacggtgatacacctattccgggctatacttatatcaaccctctcgacggcacttatccgcctggtactgagcaaaaccc
cgctaatcctaatccttctcttgaggagtctcagcctcttaatactttcatgtttcagaataataggttccgaaataggcagggggca
ttaactgtttatacgggcactgttactcaaggcactgaccccgttaaaacttattaccagtacactcctgtatcatcaaaagccatgt
atgacgcttactggaacggtaaattcagagactgcgctttccattctggctttaatgaggatccattcgtttgtgaatatcaaggcca
atcgtctgacctgcctcaacctcctgtcaatgctggcggcggctctggtggtggttctggtggcggctctgagggtggtggctctgag
ggtggcggttctgagggtggcggctctgagggaggcggttccggtggtggctctggttccggtgattttgattatgaaaagatggcaa
acgctaataagggggctatgaccgaaaatgccgatgaaaacgcgctacagtctgacgctaaaggcaaacttgattctgtcgctactga
ttacggtgctgctatcgatggtttcattggtgacgtttccggccttgctaatggtaatggtgctactggtgattttgctggctctaat
tcccaaatggctcaagtcggtgacggtgataattcacctttaatgaataatttccgtcaatatttaccttccctccctcaatcggttg
aatgtcgcccttttgtctttagcgctggtaaaccatatgaattttctattgattgtgacaaaataaacttattccgtggtgtctttgc
gtttcttttatatgttgccacctttatgtatgtattttctacgtttgctaacatactgcgtaataaggagtct
pIII Amino Acid Sequence for Wildtype M13 bacteriophage
SEQ ID NO: 60
AETVESCLAKSHTENSFTNVWKDDKTLDRYANYEGCLWNATGVVVCTGDETQCYGTWVPIGLAIPENEGGGSEGGGSEGGGSEGGGTK
PPEYGDTPIPGYTYINPLDGTYPPGTEQNPANPNPSLEESQPLNTFMFQNNRFRNRQGALTVYTGTVTQGTDPVKTYYQYTPVSSKAM
YDAYWNGKFRDCAFHSGFNEDPFVCEYQGQSSDLPQPPVNAGGGSGGGSGGGSEGGGSEGGGSEGGGSEGGGSGGGSGSGDFDYEKMA
NANKGAMTENADENALQSDAKGKLDSVATDYGAAIDGFIGDVSGLANGNGATGDFAGSNSQMAQVGDGDNSPLMNNFRQYLPSLPQSV
ECRPFVFSAGKPYEFSIDCDKINLFRGVFAFLLYVATFMYVFSTFANILRNKES
wt pIII Nucleic Acid Sequence for M13 8 + 11 bacteriophage
vector
SEQ ID NO: 61
gctgaaactgttgaaagttgtccggcaaaaccccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgaggagggtggcggttctgagggtggcggtactaaacctcctgagtac
ggtgatacacctattccgggctatacttatatcaaccctctcgacggcacttatccgcctggtactgagcaaaaccccgctaatccta
atccttctcttgaggagtctcagcctcttaatactttcatgtttcagaataataggttccgaaataggcagggggcattaactgttta
tacgggcactgttactcaaggcactgaccccgttaaaacttattaccagtacactcctgtatcatcaaaagccatgtatgacgcttac
tggaacggtaaattcagagactgcgctttccattctggctttaatgaggatttatttgtttgtgaatatcaaggccaatcgtctgacc
tgcctcaacctcctgtcaatgctggcggcggctctggtggtggttctggtggcggttctgagggtggtggctctgagggtggcggttc
tgagggtggcggctctgagggaggcggttccggtggtggctctggttccggtgattttgattatgaaaagatggcaaacgctaataag
ggggctatgaccgaaaatgccgatgaaaacgcgctacagtctgacgctaaaggcaaacttgattctgtcgctactgattacggtgctg
ctatcgatggtttcattggtgacgtttccggccttgctaatggtaatggtgctactggtgattttgctggctctaattcccaaatggc
tcaagtcggtgacggtgataattcacctttaatgaataatttccgtcaatatttaccttccctccctcaatcggttgaatgtcgccct
tttgtctttggcgctggtaaaccatatgaattttctattgattgtgacaaaataaacttattccgtggtgtctttgcgtttcttttat
atgttgccacctttatgtatgtattttcgacgtttgctaacatactgcgtaataaggagtct
(mature phage M13 surface protein P.III, encoded by
recombinant and WT g.III gene (without signal peptide))
SEQ ID NO: 62
AETVESCLAKSHTENSFTNVWKDDKTLDRYANYEGCLWNATGVVVCTGDETQCYGTWVPIGLAIPENEGGGSEGGGSEGGGSEGGGTK
PPEYGDTPIPGYTYINPLDGTYPPGTEQNPANPNPSLEESQPLNTFMFQNNRFRNRQGALTVYTGTVTQGTDPVKTYYQYTPVSSKAM
YDAYWNGKFRDCAFHSGFNEDLFVCEYQGQSSDLPQPPVNAGGGSGGGSGGGSEGGGSEGGGSEGGGSEGGGSGGGSGSGDFDYEKMA
NANKGAMTENADENALQSDAKGKLDSVATDYGAAIDGFIGDVSGLANGNGATGDFAGSNSQMAQVGDGDNSPLMNNFRQYLPSLPQSV
ECRPFVFGAGKPYEFSIDCDKINLFRGVFAFLLYVATFMYVFSTFANILRNKES
(mature, mutated phage M13 surface protein P.III
(L8P + S11P amino acid substitutions) encoded by
mutated wild-type g.III (without signal peptide))
SEQ ID NO: 63
AETVESCPAKPHTENSFTNVWKDDKTLDRYANYEGCLWNATGVVVCTGDETQCYGTWVPIGLAIPENEGGGSEGGGSEGGGSEGGGTK
PPEYGDTPIPGYTYINPLDGTYPPGTEQNPANPNPSLEESQPLNTFMFQNNRFRNRQGALTVYTGTVTQGTDPVKTYYQYTPVSSKAM
YDAYWNGKFRDCAFHSGFNEDLFVCEYQGQSSDLPQPPVNAGGGSGGGSGGGSEGGGSEGGGSEGGGSEGGGSGGGSGSGDFDYEKMA
NANKGAMTENADENALQSDAKGKLDSVATDYGAAIDGFIGDVSGLANGNGATGDFAGSNSQMAQVGDGDNSPLMNNFRQYLPSLPQSV
ECRPFVFGAGKPYEFSIDCDKINLFRGVFAFLLYVATFMYVFSTFANILRNKES
(nucleotide sequence of recombinant g.III gene (without
signal peptide-encoding sequence))
SEQ ID NO: 64
gccgagacagtggagagctgcctggccaagtcgcacaccgagaacagcttcaccaatgtttggaaggatgataagaccctggaccgct
atgccaattacgaaggttgcttatggaacgcaaccggtgtggttgtgtgcacaggcgatgagacccaatgctatggcacctgggtgcc
gatcggtctggcaattccggagaacgaaggcggaggtagcgaaggaggtggaagtgaaggcggaggatcggaagggggtggcacaaag
ccaccagaatatggagacaccccgattccaggttacacctacattaatccgctggatggtacataccctccaggcaccgaacagaatc
cggcaaacccgaacccgagcctggaagaaagccaaccgctgaacacatttatgttccaaaacaaccgttttcgtaaccgtcaaggagc
cctgaccgtatacaccggtacagtgacccagggtacagatccggtgaagacctactatcaatatacaccggttagcagcaaggcaatg
tacgatgcatattggaatggcaagtttcgtgattgtgcatttcatagcggtttcaacgaagacctgtttgtgtgcgaataccagggtc
agagcagcgatttaccgcagccaccggttaacgcaggtggtggaagcggagggggaagtggcggtgggtcagaaggcggaggatcgga
aggaggtgggagtgaaggagggggaagcgaaggagggggatcaggaggtggtagcggaagtggcgacttcgactacgagaagatggcc
aatgcaaacaaaggcgcaatgacagagaacgcagacgagaatgcactgcaaagtgatgcaaagggtaagctggacagcgttgcaaccg
actatggagcagcaattgacggctttatcggagatgtcagcggtctggcgaacggcaacggagcaacaggcgacttcgcaggtagcaa
cagccagatggcacaggttggagatggcgacaacagtccgctgatgaacaactttcgccagtacctgccgagtctgccacaaagcgtc
gagtgccgtccgtttgttttcggtgcaggcaagccgtacgagttcagcatcgactgcgataagattaatctttttcgcggagttttcg
cattcctgctgtacgtggcaacgttcatgtacgttttcagcaccttcgccaatatcttacgcaacaaagaaagc
(nucleotide sequence of mutated, wild-type g.III gene
(encoding L8P + S11P amino acid substitution)
(without signal peptide-encoding sequence))
SEQ ID NO: 65
gccgaaactgttgaaagttgtccggcaaaaccccatacagaaaattcatttactaacgtctggaaagacgacaaaactttagatcgtt
acgctaactatgagggctgtctgtggaatgctacaggcgttgtagtttgtactggtgacgaaactcagtgttacggtacatgggttcc
tattgggcttgctatccctgaaaatgagggtggtggctctgagggtggcggttctgagggtggcggttctgagggtggcggtactaaa
cctcctgagtacggtgatacacctattccgggctatacttatatcaaccctctcgacggcacttatccgcctggtactgagcaaaacc
ccgctaatcctaatccttctcttgaggagtctcagcctcttaatactttcatgtttcagaataataggttccgaaataggcagggggc
attaactgtttatacgggcactgttactcaaggcactgaccccgttaaaacttattaccagtacactcctgtatcatcaaaagccatg
tatgacgcttactggaacggtaaattcagagactgcgctttccattctggctttaatgaggatttatttgtttgtgaatatcaaggcc
aatcgtctgacctgcctcaacctcctgtcaatgctggcggcggctctggtggtggttctggtggcggctctgagggtggtggctctga
gggtggcggttctgagggtggcggctctgagggaggcggttccggtggtggctctggttccggtgattttgattatgaaaagatggca
aacgctaataagggggctatgaccgaaaatgccgatgaaaacgcgctacagtctgacgctaaaggcaaacttgattctgtcgctactg
attacggtgctgctatcgatggtttcattggtgacgtttccggccttgctaatggtaatggtgctactggtgattttgctggctctaa
ttcccaaatggctcaagtcggtgacggtgataattcacctttaatgaataatttccgtcaatatttaccttccctccctcaatcggtt
gaatgtcgcccttttgtctttggcgctggtaaaccatatgaattttctattgattgtgacaaaataaacttattccgtggtgtctttg
cgtttcttttatatgttgccacctttatgtatgtattttctacgtttgctaacatactgcgtaataaggagtct
wt pIII Amino Acid Sequence for M13 8 + 11 bacteriophage
vector
SEQ ID NO: 66
AETVESCPAKPHTENSFTNVWKDDKTLDRYANYEGCLWNATGVVVCTGDETQCYGTWVPIGLAIPENEGGGSEEGGGSEGGGTKPPEY
GDTPIPGYTYINPLDGTYPPGTEQNPANPNPSLEESQPLNTFMFQNNRFRNRQGALTVYTGTVTQGTDPVKTYYQYTPVSSKAMYDAY
WNGKFRDCAFHSGFNEDLFVCEYQGQSSDLPQPPVNAGGGSGGGSGGGSEGGGSEGGGSEGGGSEGGGSGGGSGSGDFDYEKMANANK
GAMTENADENALQSDAKGKLDSVATDYGAAIDGFIGDVSGLANGNGATGDFAGSNSQMAQVGDGDNSPLMNNFRQYLPSLPQSVECRP
FVFGAGKPYEFSIDCDKINLFRGVFAFLLYVATFMYVFSTFANILRNKES
pIII Nucleic Acid Sequence for M13 8 + 11 bacteriophage vector
SEQ ID NO: 67
gccgagacagtggagagctgcctggccaagtcgcacaccgagaacagcttcaccaatgtttggaaggatgataagaccctggaccgct
atgccaattacgaaggttgcttatggaacgcaaccggtgtggttgtgtgcacaggcgatgagacccaatgctatggcacctgggtgcc
gatcggtctggcaattccggagaacgaaggcggaggtagcgaaggaggtggaagtgaaggcggaggatcggaagggggtggcacaaag
ccaccagaatatggagacaccccgattccaggttacacctacattaatccgctggatggtacataccctccaggcaccgaacagaatc
cggcaaacccgaacccgagcctggaagaaagccaaccgctgaacacatttatgttccaaaacaaccgttttcgtaaccgtcaaggagc
cctgaccgtatacaccggtacagtgacccagggtacagatccggtgaagacctactatcaatatacaccggttagcagcaaggcaatg
tacgatgcatattggaatggcaagtttcgtgattgtgcatttcatagcggtttcaacgaagacccgtttgtgtgcgaataccagggtc
agagcagcgatttaccgcagccaccggttaacgcaggtggtggaagcggagggggaagtggcggtgggtcagaaggcggaggatcgga
aggaggtgggagtgaaggagggggaagcgaaggagggggatcaggaggtggtagcggaagtggcgacttcgactacgagaagatggcc
aatgcaaacaaaggcgcaatgacagagaacgcagacgagaatgcactgcaaagtgatgcaaagggtaagctggacagcgttgcaaccg
actatggagcagcaattgacggctttatcggagatgtcagcggtctggcgaacggcaacggagcaacaggcgacttcgcaggtagcaa
cagccagatggcacaggttggagatggcgacaacagtccgctgatgaacaactttcgccagtacctgccgagtctgccacaaagcgtc
gagtgccgtccgtacgttttcggtgcaggcaagccgtacgagttcagcatcgactgcgataagattaatctttttcgcggagttttcg
cattcctgctgtacgtggcaacgttcatgtacgttttcagcaccttcgccaatatcttacgcaacaaagaaagc
pIII Amino Acid Sequence for M13 8 + 11 bacteriophage vector
SEQ ID NO: 68
AETVESCLAKSHTENSFTNVWKDDKTLDRYANYEGCLWNATGVVVCTGDETQCYGTWVPIGLAIPENEGGGSEGGGSEGGGSEGGGTK
PPEYGDTPIPGYTYINPLDGTYPPGTEQNPANPNPSLEESQPLNTFMFQNNRFRNRQGALTVYTGTVTQGTDPVKTYYQYTPVSSKAM
YDAYWNGKFRDCAFHSGFNEDPFVCEYQGQSSDLPQPPVNAGGGSGGGSGGGSEGGGSEGGGSEGGGSEGGGSGGGSGSGDFDYEKMA
NANKGAMTENADENALQSDAKGKLDSVATDYGAAIDGFIGDVSGLANGNGATGDFAGSNSQMAQVGDGDNSPLMNNFRQYLPSLPQSV
ECRPYVFGAGKPYEFSIDCDKINLFRGVFAFLLYVATFMYVFSTFANILRNKES

The invention claimed is:

1. A modified bacteriophage pIII coat protein of the formula (from amino-terminus (N-terminus) to carboxy-terminus (C-terminus)): displayed peptide-N1-GS1-N2-GS2-CT, wherein the C-terminus of the displayed peptide is fused to the N-terminus of N1, and wherein there is a total of between 1 to 4 exogenous peptidase recognition amino acid sequences within GS1 and GS2 of the pIII coat protein, and wherein at least one exogenous peptidase recognition amino acid sequence is FLVIR (SEQ ID NO: 4).

2. (canceled)

3. The modified pIII coat protein of claim 1, wherein the bacteriophage is a M13 bacteriophage.

4. The modified pIII coat protein of claim 3, wherein the M13 bacteriophage is otherwise encoded by a nucleic acid sequence shown in SEQ ID NOs: 1, 2, or 57.

5. The modified pIII coat protein of claim 3, wherein there is a total of between 1 to 3 exogenous peptidase recognition amino acid sequences within GS1 and GS2 of the pIII coat protein, and wherein at least one exogenous peptidase recognition amino acid sequence is FLVIR (SEQ ID NO: 4).

6. The modified pIII coat protein of claim 4, wherein there is a total of between 1 to 3 exogenous peptidase recognition amino acid sequences within GS1 and GS2 of the pIII coat protein, and wherein at least one exogenous peptidase recognition amino acid sequence is FLVIR (SEQ ID NO: 4).

7. The modified pIII coat protein of claim 5, wherein there is a total of two exogenous peptidase recognition amino acid sequences within GS1 and GS2 of the pIII coat protein, and wherein at least one exogenous peptidase recognition amino acid sequence is FLVIR (SEQ ID NO: 4).

8. The modified pIII coat protein of claim 6, wherein there is a total of two exogenous peptidase recognition amino acid sequences within GS1 and GS2 of the pIII coat protein, and wherein at least one exogenous peptidase recognition amino acid sequence is FLVIR (SEQ ID NO: 4).

9. (canceled)

10. (canceled)

11. The modified pIII coat protein of claim 7, wherein one exogenous peptidase recognition amino acid sequence FLVIR (SEQ ID NO: 4) is inserted into the GS2 linker of the pIII coat protein.

12. The modified pIII coat protein of claim 8, wherein the exogenous peptidase recognition amino acid sequence FLVIR (SEQ ID NO: 4) is inserted into the GS2 linker of the pIII coat protein.

13. The modified pIII coat protein of claim 11 wherein the displayed peptide is either a cell-penetrating peptide (CPP) or a putative CPP.

14. The modified pIII coat protein of claim 12 wherein the displayed peptide is either a cell-penetrating peptide (CPP) or a putative CPP.

15. A bacteriophage comprising the modified pIII coat protein of claim 3.

16. A bacteriophage comprising the modified pIII coat protein of claim 4.

17. A bacteriophage comprising the modified pIII coat protein of claim 14.

18. A bacteriophage library comprising a plurality of bacteriophage of claim 17.

19. The bacteriophage library of claim 18, wherein the modified pIII coat protein comprises an amino acid sequence encoded by a nucleic acid selected from the group consisting of SEQ ID NOs: 49-56.

20. A method of making a bacteriophage having a modified pIII coat protein, comprising the step of:
(a) modifying a pIII coat protein of a bacteriophage to comprise a total of between 1 to 4 exogenous peptidase recognition amino acid sequences within GS1 and GS2 of the pIII coat protein, wherein at least one exogenous peptidase recognition amino acid sequence is FLVIR (SEQ ID NO: 4), and
(b) obtaining the bacteriophage having the modified pIII coat protein of the formula (from amino-terminus (N-terminus) to carboxy-terminus (C-terminus)): displayed peptide-N1-GS1-N2-GS2-CT, wherein the C-terminus of the displayed peptide is fused to the N-terminus of N1.

21. The method of claim 20, wherein at least one exogenous peptide recognition amino acid sequence FLVIR (SEQ ID NO: 4) is inserted into both the GS1 linker and the GS2 linker of the modified pIII coat protein.

22. The method of claim 21, wherein one exogenous peptidase amino acid sequence FLVIR (SEQ ID NO: 4) is inserted into the GS1 linker of the modified pIII coat protein.

23. The method of claim 22, wherein one exogenous peptidase amino acid sequence FLVIR (SEQ ID NO: 4) is inserted into the GS2 linker of the modified pIII coat protein.

24. The method of claim 22, wherein the modified pIII comprises an amino acid sequence encoded by a nucleic acid sequence selected from the group consisting of SEQ ID NOs: 49-56.

25. The method of claim 24 wherein the displayed peptide is a CPP or a putative CPP.

26. A method of screening bacteriophage library for clones that avoid lysosomal compartments, the method comprising the steps of:
providing a bacteriophage library of claim 18;
exposing the bacteriophage library to a target cell population for a predetermined period of time to obtain internalized bacteriophage;
washing the target cell population to remove uninternalized bacteriophage and to obtain a washed target cell population;
lysing the washed target cell population and obtaining recovered internalized bacteriophage; and
identifying the recovered bacteriophage as clones that avoid lysosomal compartments in the target cell population.

27. A method of screening a bacteriophage library for clones that avoid lysosomal compartments, the method comprising the steps of:
providing a bacteriophage library of claim 19;
exposing the bacteriophage library to a target cell population for a predetermined period of time to obtain internalized bacteriophage;
washing the target cell population to remove uninternalized bacteriophage and to obtain a washed target cell population;
lysing the washed target cell population and obtaining recovered internalized bacteriophage; and
identifying the recovered bacteriophage as clones that avoid lysosomal compartments in the target cell population.

28. The method of claim 27 further comprising the step of:
amplifying the recovered internalized bacteriophage prior to the step of identifying the recovered bacteriophage as clones that avoid lysosomal compartments in the target cell population.

29. (canceled)

30. The method of claim 28, wherein the target cell population is a mammalian cell population.

31. The method of claim 30, wherein the mammalian cell population is selected from the group consisting of pancreatic beta cells, adipocytes, alveolar epithelium cells, fibroblasts, skeletal muscle cells, cardiomyocytes, CHO cells, 293 cells, CaCo2 cells, or neurons, including, but not limited to, dorsal root ganglion (DRG) neurons, and hypothalamic neurons.

32. A method of screening a bacteriophage or a bacteriophage library for bacteriophages that are sensitive to lysosomal enzymes, the method comprising the steps of:
providing the bacteriophage library of claim 18;
exposing the bacteriophage library to a lysosomal enzyme for a predetermined period of time to obtain cleaved bacteriophages and uncleaved bacteriophages; and
identifying bacteriophages that are cleaved by the lysosomal enzyme.

33. A method of screening a bacteriophage or a bacteriophage library for bacteriophages that are sensitive to lysosomal enzymes, the method comprising the steps of:
providing the bacteriophage library of claim 19;
exposing the bacteriophage library to a lysosomal enzyme for a predetermined period of time to obtain cleaved bacteriophages and uncleaved bacteriophages; and
identifying bacteriophages that are cleaved by the lysosomal enzyme.

34. The method of claim 33, wherein the lysosomal enzyme is a cathepsin.

35. (canceled)

36. A method of screening putative cell-penetrating peptides (CPPs), the method comprising the steps of:
providing the bacteriophage library of claim 18;
exposing the bacteriophage library to a first target cell population for a predetermined period of time to obtain internalized engineered bacteriophage;
washing the first target cell population to remove uninternalized bacteriophage and to obtain a washed target cell population;
lysing the washed first target cell population and obtaining recovered internalized bacteriophage;
exposing the recovered internalized bacteriophage to a second target cell population for a predetermined period of time to infect the second target cell population and to obtain amplified, recovered internalized bacteriophage; and
identifying the amplified, recovered bacteriophage for clones that avoided lysosomal compartments in the first target cell population.

37. A method of screening putative cell-penetrating peptides (CPPs), the method comprising the steps of:
providing the bacteriophage library of claim 19;
exposing the bacteriophage library to a first target cell population for a predetermined period of time to obtain internalized bacteriophage;
washing the first target cell population to remove uninternalized bacteriophage and to obtain a washed target cell population;
lysing the washed first target cell population and obtaining recovered internalized bacteriophage;
exposing the recovered internalized bacteriophage to a second target cell population for a predetermined period of time to infect the second target cell population and to obtain amplified, recovered internalized bacteriophage; and
identifying the amplified, recovered bacteriophage for clones that avoided lysosomal compartments in the first target cell population.

38. (canceled)

39. The method of claim 37, the first target cell population is a mammalian cell population.

40. The method of claim 39, wherein the mammalian cell population is selected from the group consisting of pancreatic beta cells, adipocytes, alveolar epithelium cells, fibroblasts, skeletal muscle cells, cardiomyocytes, CHO cells, 293 cells, CaCo2 cells, or neurons, including, but not limited to, dorsal root ganglion (DRG) neurons, and hypothalamic neurons.

41. (canceled)

42. A compound comprising: 1) a CPP identified through the use of the method of claim 40; and 2) a peptide, protein, LNP, a PLV, mRNA, iRNA, siRNA, ASO, mAb fragment or a small molecule.
